(19) United States
(12) Patent Application Publication
(10) Pub. No.: US 2026/0074056 A1
(43) Pub. Date: Mar. 12, 2026

(54) SYSTEMS AND METHODS FOR MEDICAL IMAGING

(71) Applicant: SHANGHAI UNITED IMAGING HEALTHCARE CO., LTD., Shanghai (CN)

(72) Inventors: Jing YAN, Shanghai (CN); Juan FENG, Shanghai (CN); Jian ZHONG, Shanghai (CN)

(73) Assignee: SHANGHAI UNITED IMAGING HEALTHCARE CO., LTD., Shanghai (CN)

(21) Appl. No.: 19/309,576

(22) Filed: Aug. 25, 2025

(30) Foreign Application Priority Data
Sep. 6, 2024 (CN) 202411254745.0
Nov. 6, 2024 (CN) 202411581198.7
Dec. 6, 2024 (CN) 202411800360.X

(51) Int. Cl. G16H 30/40

(52) U.S. Cl. CPC G16H 30/40

(57) ABSTRACT

The present disclosure provides a system and method for medical imaging. The method includes obtaining a first reconstructed image of a target subject; and generating a target reconstructed image of the target subject by inputting the first reconstructed image into a trained deep learning model. An image quality of the target reconstructed image is higher than that of the first reconstructed image.

### CROSS-REFERENCE TO RELATED APPLICATIONS

This application claims the priority to Chinese Patent Application No. 202411254745.0, filed on Sep. 6, 2024, Chinese Patent Application No. 202411581198.7, filed on Nov. 6, 2024, and Chinese Patent Application No. 202411800360.X, filed on Dec. 6, 2024, the contents of each of which are hereby incorporated by reference.

### TECHNICAL FIELD

The present disclosure generally relates to the field of medical imaging, and more particularly relates to a method and a system for medical imaging.

### BACKGROUND

Perfusion imaging technology is an important means for medical diagnosis. Perfusion imaging technology utilizes the flow and distribution process of a contrast agent within tissues to observe changes in local blood flow and vascular permeability, thereby assessing the supply and demand of blood in tissues. During perfusion imaging, a contrast agent can be injected into a patient, and continuous CT scans are performed to capture the process of the contrast agent circulating within the body, allowing for the assessment of tissue blood perfusion.

By obtaining a time attenuation curve of tissues and blood vessels from images acquired through perfusion scanning imaging, and generating a perfusion parameter map based on the time attenuation curve, potential salvageable ischemic tissues that may undergo reperfusion through catheter-guided stroke therapy can be identified.

Cone beam computed tomography (CBCT) is a commonly used perfusion scanning imaging method in perfusion imaging. In CBCT perfusion imaging, a plurality of time frames are reconstructed to determine a time-density curve for estimating perfusion information. A count (sampling count), temporal resolution, and image quality of the plurality of time frames are crucial for determining the time-density curve.

Therefore, it is desired to provide an imaging method and system that can effectively improve the sampling count, temporal resolution, and image quality of the time frames.

### SUMMARY

According to a first aspect of the present disclosure, a method for medical imaging is provided. The method may be implemented on at least one computing device, each of which may include at least one processor and a storage device. The method may include obtaining a first reconstructed image of a target subject; and generating a target reconstructed image of the target subject by inputting the first reconstructed image into a trained deep learning model, wherein an image quality of the target reconstructed image is higher than that of the first reconstructed image. The trained deep learning model includes a plurality of primary feature extraction blocks that are connected in series and a plurality of secondary feature extraction blocks. An input of a first primary feature extraction block of the plurality of primary feature extraction blocks includes the first reconstructed image. An input of each of the primary feature extraction blocks other than the first primary feature extraction block includes an output of a previous primary feature extraction block. An output of a last primary feature extraction block of the plurality of primary feature extraction blocks includes a first sub-feature map. For each of the plurality of secondary feature extraction blocks, an input of the secondary feature extraction block includes the input of one of the plurality of primary feature extraction blocks, and an output of the secondary feature extraction block includes a second sub-feature map. The target reconstructed image is determined based on the first sub-feature map and the second sub-feature maps output by the plurality of secondary feature extraction blocks.

### BRIEF DESCRIPTION OF THE DRAWINGS

The present disclosure is further described in terms of exemplary embodiments. These exemplary embodiments are described in detail with reference to the drawings. The drawings are not scaled. These embodiments are non-limiting exemplary embodiments, in which like reference numerals represent similar structures throughout the several views of the drawings, and wherein:

FIG. 1 is a schematic diagram of an exemplary application scenario of a medical imaging system according to some embodiments of the present disclosure;

FIG. 2 is a structural schematic diagram of an exemplary C-arm machine according to some embodiments of the present disclosure;

FIG. 3 is a block diagram of an exemplary medical imaging system according to some embodiments of the present disclosure;

FIG. 4 is a flowchart illustrating an exemplary process for medical imaging according to some embodiments of the present disclosure;

FIG. 5 is a schematic diagram illustrating an exemplary logical structure of a deep learning model according to some embodiments of the present disclosure;

FIG. 6 is a schematic diagram illustrating an exemplary logical structure of a primary feature extraction block according to some embodiments of the present disclosure;

FIG. 7 is a schematic diagram illustrating an exemplary logical structure of a secondary feature extraction block according to some embodiments of the present disclosure;

FIG. 8A is a schematic diagram illustrating an exemplary process for training a deep learning model according to some embodiments of the present disclosure;

FIG. 8B is a schematic diagram illustrating an exemplary process for training a deep learning model according to some embodiments of the present disclosure;

FIG. 9A is a block diagram of an exemplary medical imaging system according to some embodiments of the present disclosure;

FIG. 9B is a flowchart illustrating an exemplary process for obtaining a first reconstructed image according to some embodiments of the present disclosure;

FIG. 10A-FIG. 10C are schematic diagrams illustrating exemplary full-angle scanning and rotation modes of an imaging device according to some embodiments of the present disclosure;

FIG. 10D is a schematic diagram illustrating an exemplary process for generating a third reconstructed image according to some embodiments of the present disclosure;

FIG. 10E is a schematic diagram illustrating an exemplary process for generating a third reconstructed image according to some embodiments of the present disclosure;

FIG. 10F is a schematic diagram illustrating exemplary correction scanning data according to some embodiments of the present disclosure;

FIG. 11 is a flowchart illustrating an exemplary process for obtaining a first reconstructed image according to some embodiments of the present disclosure;

FIG. 12A is a block diagram illustrating an exemplary medical imaging system according to some embodiments of the present disclosure;

FIG. 12B is a flowchart illustrating an exemplary process for obtaining a first reconstructed image according to some embodiments of the present disclosure;

FIG. 13A is a schematic diagram illustrating an exemplary scanning process of an imaging device according to some embodiments of the present disclosure;

FIG. 13B is a schematic diagram illustrating an exemplary contiguous projection angle range according to some embodiments of the present disclosure;

FIG. 13C is a schematic diagram illustrating an exemplary ring-shaped detector according to some embodiments of the present disclosure;

FIG. 13D is a schematic diagram illustrating an exemplary arc-shaped detector according to some embodiments of the present disclosure;

FIG. 14A is a schematic diagram illustrating an exemplary overlapping projection angle range according to some embodiments of the present disclosure;

FIG. 14B is a schematic diagram illustrating an exemplary projection angle range with a gap according to some embodiments of the present disclosure;

FIG. 15 is a flowchart illustrating an exemplary process for obtaining a target reconstructed image according to some embodiments of the present disclosure; and

FIG. 16 is an exemplary structural diagram of an exemplary computer device according to some embodiments of the present disclosure.

### DETAILED DESCRIPTION

To more clearly illustrate the technical solutions in the embodiments of the present disclosure, the drawings used in the description of the embodiments will be briefly introduced below. Obviously, the drawings in the following description are merely some examples or embodiments of the present disclosure. For those of ordinary skill in the art, without creative effort, the present disclosure can be applied to other similar scenarios based on these drawings. Unless obvious from the context or otherwise stated, identical reference numerals in the figures denote identical structures or operations.

The terms “system,” “device,” “unit,” and/or “module” used herein are methods for distinguishing different components, elements, parts, sections, or assemblies at different levels. However, if other words can achieve the same purpose, said words may be replaced by other expressions.

Unless the context clearly dictates an exception, the terms “a,” “an,” “the,” and the like are not intended to be singular and may include the plural. Generally, the terms “include” and “comprise” only suggest the inclusion of explicitly identified steps and elements, and these steps and elements do not constitute an exclusive list. Methods or devices may also contain other steps or elements.

The flowcharts used in the present disclosure illustrate operations that systems implement according to some embodiments in the present disclosure. It is to be expressly understood, the operations of the flowchart may be implemented not in order. Conversely, the operations may be implemented in an inverted order or simultaneously. Moreover, one or more other operations may be added to the flowcharts. One or more operations may be removed from the flowcharts.

Some embodiments of the present disclosure provide a medical imaging method. A plurality of sets of projection data from a plurality of projection angle ranges are simultaneously acquired using a plurality of radiation sources (e.g., at least two radiation sources). The plurality of sets of projection data are combined to generate combined projection data. A medical image is generated by reconstructing the combined projection data. By simultaneously acquiring the plurality of sets of projection data from the plurality of projection angle ranges using the plurality of radiation sources (e.g., at least two radiation sources), the scanning time required to obtain projection data for generating one image is significantly reduced. For static imaging, this improves image generation efficiency. For dynamic imaging, this improves the temporal resolution and temporal sampling rate of dynamic images.

Some embodiments of the present disclosure provide an image reconstruction method, including: obtaining a plurality of sets of initial scanning data, each of the plurality of sets of initial scanning data being acquired by performing a circle of full-angle scanning on a target subject using an imaging device; for each of the plurality of sets of initial scanning data, generating initial reconstruction data by performing reconstruction based on the set of initial scanning data; generating a third reconstructed image corresponding to a target time point by performing, in a chronological order, interpolating based on the initial reconstruction data of the plurality of sets of initial scanning data; and generating a first reconstructed image (or a limited-angle reconstructed image) corresponding to the target time point based on the third reconstructed image and a portion (i.e., correction scanning data) of the plurality of sets of initial scanning data. The correction scanning data may refer to the initial scanning data at the target time point and initial scanning data within a time period adjacent to the target time point among the plurality of sets of initial scanning data. By reconstructing based on the initial scanning data at the target time point and initial scanning data within the time period adjacent to the target time point, an amount of correction scanning data required to obtain the first reconstructed image is smaller. Therefore, reconstruction efficiency is improved while enhancing the temporal resolution and temporal sampling rate. Additionally, initial scanning data closer to the target time point more accurately reflects a true attenuation value at the target time point. Thus, reconstruction based on data at the target time point and data within the time period adjacent to the target time point can further improve the image quality of the resulting first reconstructed image.

Due to the aforementioned limited-angle reconstructed image is reconstructed using limited-angle initial scanning data, significant artifacts (i.e., limited-angle artifacts) may be present. In order to eliminate the limited-angle artifacts of the limited-angle reconstructed image, some embodiments of the present disclosure provide a medical imaging method, including: inputting a limited-angle reconstructed image into a deep learning model to obtain an artifact-optimized reconstructed image (i.e., a target reconstructed image). The deep learning model includes: a plurality of primary feature extraction blocks that are connected in series, wherein an input of a first primary feature extraction block of the plurality of primary feature extraction blocks includes the first reconstructed image, an input of each of the primary feature extraction blocks other than the first primary feature extraction block includes an output of a previous primary feature extraction block, and an output of a last primary feature extraction block of the plurality of primary feature extraction blocks includes a first sub-feature map; and a plurality of secondary feature extraction blocks, wherein for each of the plurality of secondary feature extraction blocks, an input of the secondary feature extraction block includes the input of one of the plurality of primary feature extraction blocks, and an output of the secondary feature extraction block includes a second sub-feature map; wherein the target reconstructed image is determined based on the first sub-feature map and the second sub-feature maps output by the plurality of secondary feature extraction blocks. The deep learning model outputs the target reconstructed image by performing N iterations in a reverse order based on the first sub-feature map and the second sub-feature maps output by the plurality of secondary feature extraction blocks. Through the structure of the deep learning model and the multi-iteration processing performed by the deep learning model based on its structure, the features extracted by the deep learning model can be optimized, leading to better artifact removal effects on the limited-angle reconstructed image.

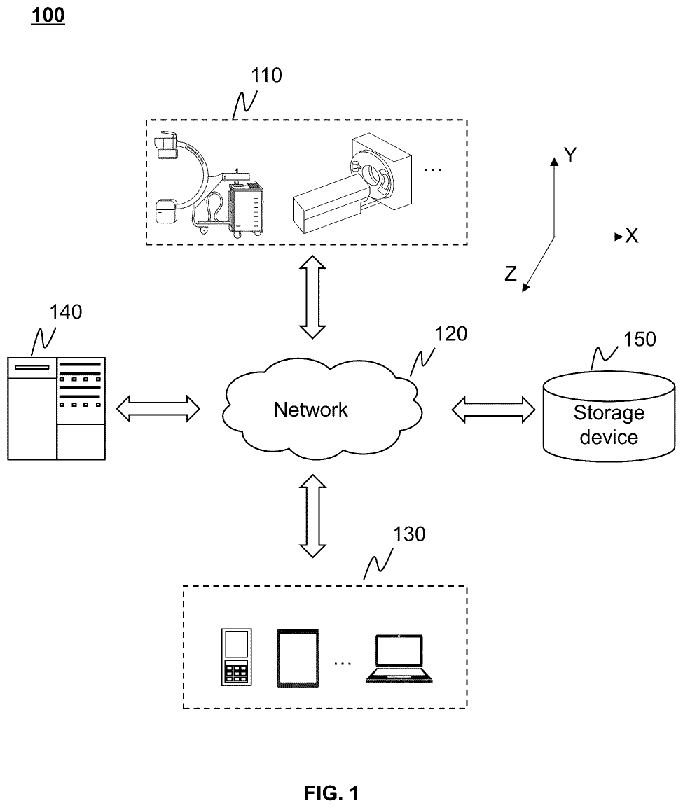

FIG. 1 is a schematic diagram of an exemplary application scenario of a medical imaging system according to some embodiments of the present disclosure.

As shown in FIG. 1, an application scenario 100 involving a medical system of the embodiments of the present disclosure includes an imaging device 110, a network 120, a terminal device 130, a processing device 140, and a storage device 150. The X-axis, Y-axis, and Z-axis shown in FIG. 1 form an orthogonal coordinate system. The Z-axis is horizontal, with its positive direction in FIG. 1 pointing towards a direction for moving a target subject out of a scanning region of the imaging device 110. The X-axis shown in FIG. 1 is horizontal, and the Y-axis shown in FIG. 1 is vertical. As shown in FIG. 1, a positive direction of the X-axis points from the left side to the right side of the imaging device 110 when viewed along a negative direction of the Z-axis. A positive direction of the Y-axis points from a lower part to an upper part of the imaging device 110.

The imaging device 110 may scan a target subject within a detection region or a scanning region to obtain scanning data of the target subject.

The target subject is an object undergoing lesion detection. The target subject may be a patient, an experimental subject, etc. In some embodiments, the target subject may include biological and/or non-biological objects. For example, the target subject may include a patient, a man-made object, etc. In some embodiments, the target subject may include specific parts of the body, such as the head, chest, abdomen, or the like, or any combination thereof. In some embodiments, the target subject may include specific organs, such as the heart, esophagus, trachea, bronchi, stomach, gallbladder, small intestine, colon, bladder, ureter, uterus, fallopian tubes, or the like, or any combination thereof. In some embodiments, the target subject may include a region of interest (ROI), such as a tumor, lymph nodes, soft tissue, etc.

In some embodiments, the imaging device 110 may be an imaging device that utilizes radiation rays for imaging. The radiation rays include particle rays, photon rays, etc. The particle rays may include neutrons, protons, electrons, muons, heavy ions, alpha rays, or any combination thereof. The photon rays may include X-rays, gamma rays, ultraviolet light, lasers, or any combination thereof. For example, the imaging device 110 may include a digital subtraction angiography (DSA) imaging device, a CT device (e.g., a fan-beam CT, a cone-beam CT, etc.), etc. In some embodiments, the imaging device 110 may include a single-modality scanner and/or a multi-modality scanner. The single-modality scanner may include, for example, a CT scanner, a DSA scanner, etc. The multi-modality scanner may include, for example, a computed tomography-magnetic resonance imaging (CT-MRI) scanner, a single-photon emission computed tomography-computed tomography (SPECT-CT) scanner, a positron emission tomography-computed tomography (PET-CT) scanner, a digital subtraction angiography-magnetic resonance imaging (DSA-MRI) scanner, or the like, or any combination thereof. The above description of the imaging device 110 is for illustrative purposes only and is not intended to limit the scope of the present disclosure.

In some embodiments, the imaging device 110 may be applied in an imaging system. In some embodiments, the imaging device 110 may be applied in a therapy system. For example, the therapy system may include an image-guided radiotherapy (IGRT) device. The IGRT device includes a therapy component and an imaging component. The therapy component includes a linear accelerator, a cyclotron, a synchrotron, etc. The therapy component is configured to deliver radiation therapy to the target subject. The imaging component includes the imaging device 110, an electronic portal imaging device (EPID), etc.

In some embodiments, the imaging device 110 includes at least one radiation source and at least one detector.

The radiation source is a component that generates scanning rays (e.g., X-rays). Taking an X-ray source as an example, the radiation source includes a high-voltage generator and an X-ray tube. The high-voltage generator provides high voltage to accelerate electrons, and the X-ray tube is where the electrons strike a metal target to produce X-rays. The radiation source may emit a fan-shaped or cone-shaped beam of radiation.

The detector is configured to receive rays generated by the radiation source. The rays are first converted into visible light (e.g., scintillation crystals precisely capture X-ray photons and produce fluorescence). The visible light is converted into an electrical signal (e.g., by photodiodes). The electrical signal is converted into a digital signal (e.g., by analog-to-digital converters), and the digital signal is input into a computer (e.g., the processing device 140) for processing (e.g., image reconstruction). The digital signal may be referred to as projection data or initial scanning data.

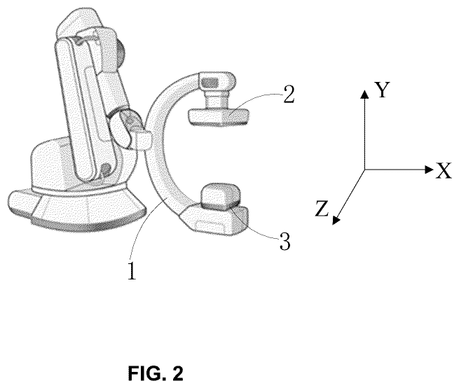

Exemplarily, the imaging device 110 may be a C-arm machine as shown in FIG. 2, including a C-arm 1, a detector 2, and a tube (i.e., the radiation source) 3. The C-arm 1 supports the detector 2 and the tube 3. The target subject is placed between the detector 2 and the tube 3. The beam emitted by the tube 3 passes through the target subject and is incident on the detector 2. As another example, the radiation source and the detector may be mounted on a ring gantry (e.g., as shown in FIG. 1). During imaging scanning, the target subject is placed within a scanning cavity of the ring gantry.

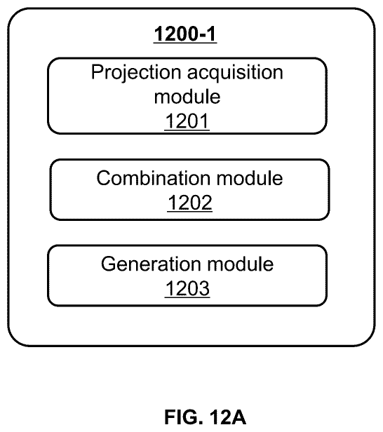

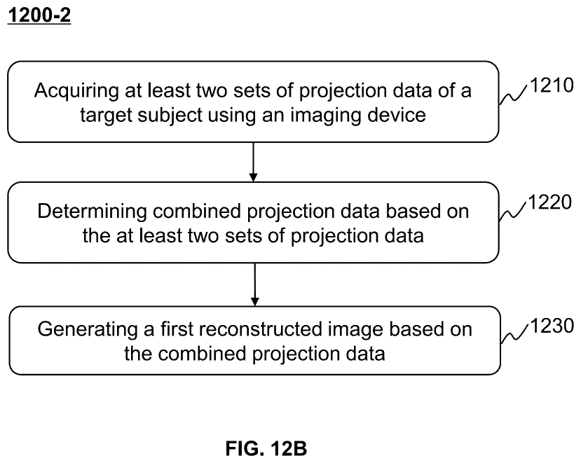

In some embodiments, the imaging device may include at least two radiation sources and at least one detector. Specific descriptions are provided in FIGS. 12A-14.

In some embodiments, the imaging device 110 further includes a contrast agent injector. The contrast agent injector is configured to inject a contrast agent. The contrast agent injector includes an injector body, a high-pressure pump, and other components. The injector body holds the contrast agent, and the high-pressure pump generates pressure to push the contrast agent into the patient's body.

The network 120 may include any suitable network facilitating information and/or data exchange within the application scenario 100. In some embodiments, one or more components of the application scenario 100 (e.g., the imaging device 110, the terminal device 130, the processing device 140, the storage device 150, etc.) may transmit information and/or data to one or more other components of the application scenario 100 via the network 120. For example, the processing device 140 may obtain projection data from the imaging device 110 via the network 120. In some embodiments, the network 120 may be any one or more of a wired or wireless network. In some embodiments, the network 120 may have various topologies such as point-to-point, shared, centralized, or the like, or any combination thereof.

The terminal device 130 refers to one or more terminal devices or software used by a user to initiate a scanning instruction or receive an artifact-optimized reconstructed image generated by the processing device 140. The user may refer to personnel who analyze and use the application scenario 100, such as a doctor, a medical researcher, etc. The terminal device 130 may include a mobile device, a tablet computer, a laptop computer, or the like, or any combination thereof. In some embodiments, the terminal device 130 may interact with other components in the application scenario 100 via the network 120. For example, the terminal device 130 may receive projection data sent by the imaging device 110. In some embodiments, the terminal device 130 may receive information and/or instructions input by a user (e.g., an operator of the imaging device 110, such as a doctor) and send the received information and/or instructions to the imaging device 110 or the processing device 140 via the network 120. For example, a doctor may input an operation instruction for the imaging device 110 via the terminal device 130. In some embodiments, the terminal device 130 may display an imaging result (e.g., a generated image). In some embodiments, the terminal device 130 may also be integrated with the imaging device 110.

The processing device 140 may process data and/or information obtained from the imaging device 110, the terminal device 130, and/or the storage device 150.

In some embodiments, the processing device 140 may be a single server or a server group. The server group may be centralized or distributed. In some embodiments, the processing device 140 may be local or remote. The processing device 140 may be directly connected to the imaging device 110, the terminal device 130, and the storage device 150 to access stored or acquired information and/or data. In some embodiments, the processing device 140 may be implemented on a cloud platform. Merely by way of example, the cloud platform may include a private cloud, a public cloud, a hybrid cloud, a community cloud, a distributed cloud, an intercloud, a multi-cloud, or the like, or any combination thereof.

The storage device 150 may store data and/or instructions. In some embodiments, the storage device 150 may store data acquired from the imaging device 110, the terminal device 130, and/or the processing device 140. For example, the storage device 150 may store projection data acquired by the imaging device 110. In some embodiments, the storage device 150 may store data and/or instructions used by the processing device 140 to execute the exemplary methods described in the present disclosure. In some embodiments, the storage device 150 may include a mass storage device, a removable storage device, a volatile read-write memory, a read-only memory (ROM), etc., or any combination thereof. In some embodiments, the storage device 150 may be implemented on a cloud platform.

In some embodiments, the storage device 150 may be connected to the network 120 to communicate with one or more components of the application scenario 100 (e.g., the imaging device 110, the terminal device 130, the processing device 140, etc.). One or more components of the application scenario 100 may access data or instructions stored in the storage device 150 via the network 120. In some embodiments, the storage device 150 may be directly connected to or communicate with one or more components of the application scenario 100. In some embodiments, the storage device 150 may be part of the processing device 140.

It should be noted that the application scenario 100 is provided solely for illustrative purposes and is not intended to limit the scope of the present disclosure. Those of ordinary skill in the art may make various modifications or changes based on the description in the present disclosure. For example, the terminal device 130 may also include other types of terminals. However, these changes and modifications do not depart from the scope of the present disclosure.

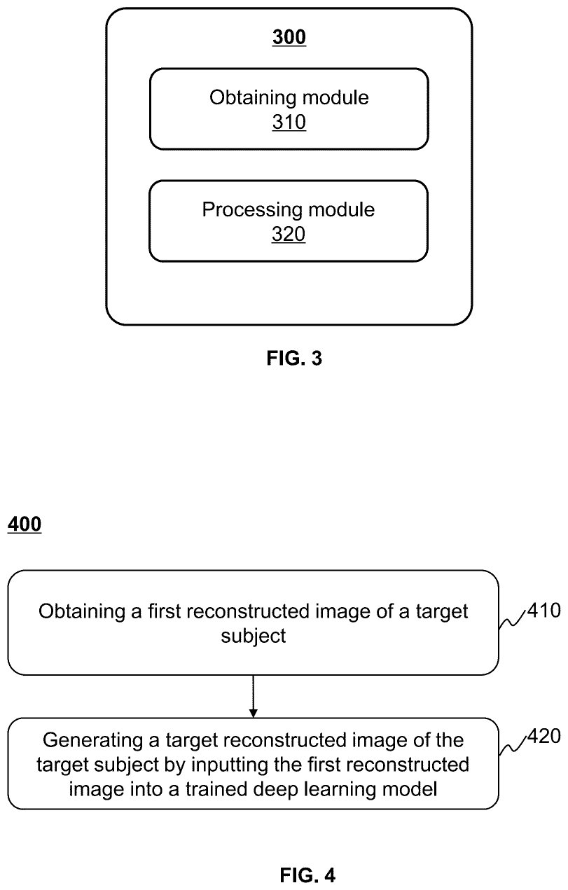

FIG. 3 is a block diagram of an exemplary medical imaging system according to some embodiments of the present disclosure. In some embodiments, as shown in FIG. 3, a medical imaging system 300 includes an obtaining module 310 and a processing module 320. The medical imaging system 300 may be implemented on the processing device 140 through hardware or software.

The obtaining module 310 is configured to obtain a first reconstructed image of a target subject. For example, the obtaining module 310 may acquire initial scanning data from the imaging device 110.

The processing module 320 includes a deep learning model. The processing module 320 is configured to generate a target reconstructed image of the target subject by processing the first reconstructed image using the deep learning model.

More descriptions regarding the first reconstructed image, the deep learning model, and the target reconstructed image can be found in FIGS. 4 to 8B and their related descriptions.

It should be noted that the above description of the medical imaging system 300 and its modules is for convenience of description only and should not limit the present disclosure to the scope of the illustrated embodiments. It is understood that for those skilled in the art, after understanding the principles of the system, they may combine the modules arbitrarily or form subsystems connected to other modules without departing from these principles. In some embodiments, the obtaining module 310 and the processing module 320 disclosed in FIG. 3 may be different modules within one system, or one module may implement the functions of two or more of the above modules. For example, the modules may share a storage module, or the modules may each have their own respective storage modules. Such variations are all within the scope of the present disclosure.

FIG. 4 is a flowchart illustrating an exemplary process for medical imaging according to some embodiments of the present disclosure. As shown in FIG. 4, process 400 includes the following operations. In some embodiments, process 400 may be performed by the medical imaging system 300 and/or the processing device 140.

In 410, the processing device 140 obtains a first reconstructed image of a target subject.

More descriptions regarding the target subject can be found in FIG. 1 and its related description.

The first reconstructed image refers to an image obtained by preliminary reconstruction. The first reconstructed image may be a two-dimensional (2D) image or a three-dimensional (3D) image. The first reconstructed image may be a dynamic image including a plurality of time frames or a single static image. The first reconstructed image may include artifacts, such as metal artifacts, motion artifacts, limited-angle artifacts, etc. The present disclosure does not limit the type of artifacts in the first reconstructed image. The first reconstructed image may include an MRI image, a CT image, a PET image, an X-ray image, etc. The present disclosure does not limit the type of the first reconstructed image.

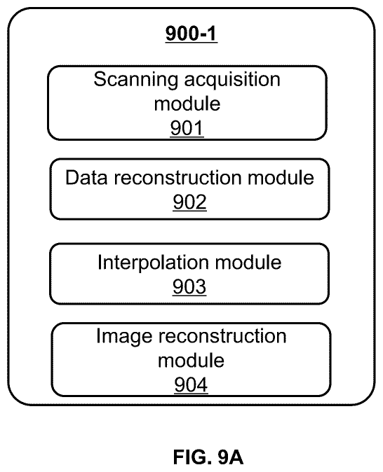

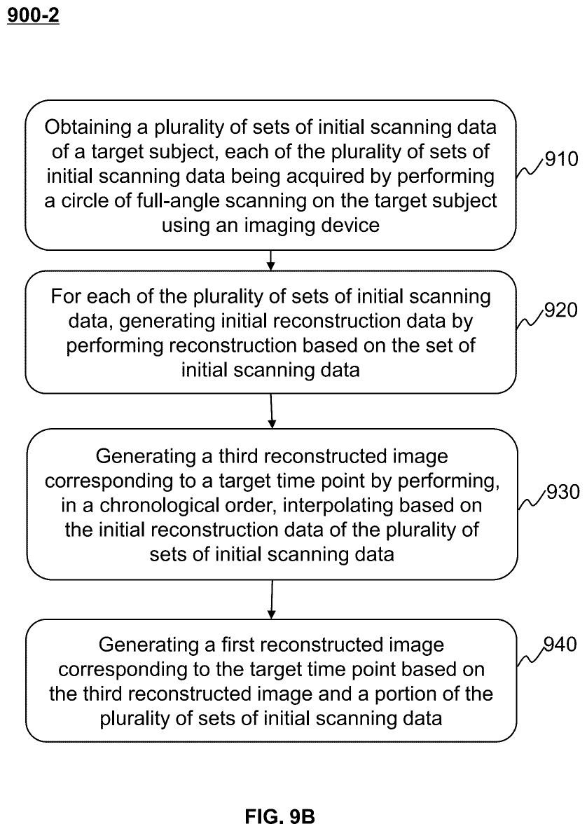

In some embodiments, the first reconstructed image may be a limited-angle reconstructed image. The limited-angle reconstructed image refers to an image obtained by reconstruction based on projection data from a limited range of projection angles. More descriptions regarding generating the limited-angle reconstructed image can be found in FIGS. 9A-11 and their related descriptions.

In 420, the processing device 140 generates a target reconstructed image of the target subject by inputting the first reconstructed image into a trained deep learning model. In some embodiments, a deep learning model 500 described herein may be used to improve the image quality of medical images. Further, the deep learning model 500 may be used to eliminate or suppress artifacts (e.g., metal artifacts, motion artifacts, limited-angle artifacts, etc.) in medical images (e.g., MRI images, CT images, PET images, X-ray images, etc.).

The target reconstructed image may be an image obtained by eliminating artifacts (e.g., motion artifacts, metal artifacts, limited-angle artifacts), or an image where original artifacts in the first reconstructed image are sufficiently suppressed.

Artifacts being sufficiently suppressed indicates that a residual artifact amount in the target reconstructed image is less than a residual threshold. The residual artifact amount refers to a ratio of an artifact area in the target reconstructed image to an artifact area in the first reconstructed image (i.e., an image not optimized for artifacts). The artifact area may be characterized based on a count of pixels occupied by artifacts. For example, if the artifact area in the target reconstructed image is 200 pixels, and the artifact area in the first reconstructed image is 5000 pixels, the residual artifact amount of the target reconstructed image is (200/5000)×100%=4%. The residual threshold may be preset, e.g., 3%, 5%, 7%, 10%, etc. It should be understood that when artifacts in the target reconstructed image are sufficiently suppressed (i.e., the residual artifact amount is below the residual threshold), the residual artifacts in the target reconstructed image may not affect subsequent diagnostic results obtained based on the target reconstructed image.

In some embodiments, an image quality of the target reconstructed image is higher than that of the first reconstructed image.

The deep learning model is a machine learning model. In some embodiments, the deep learning model is configured to process the first reconstructed image to obtain the target reconstructed image. In this embodiment, an input of the deep learning model includes the first reconstructed image, and an output of the deep learning model includes the target reconstructed image.

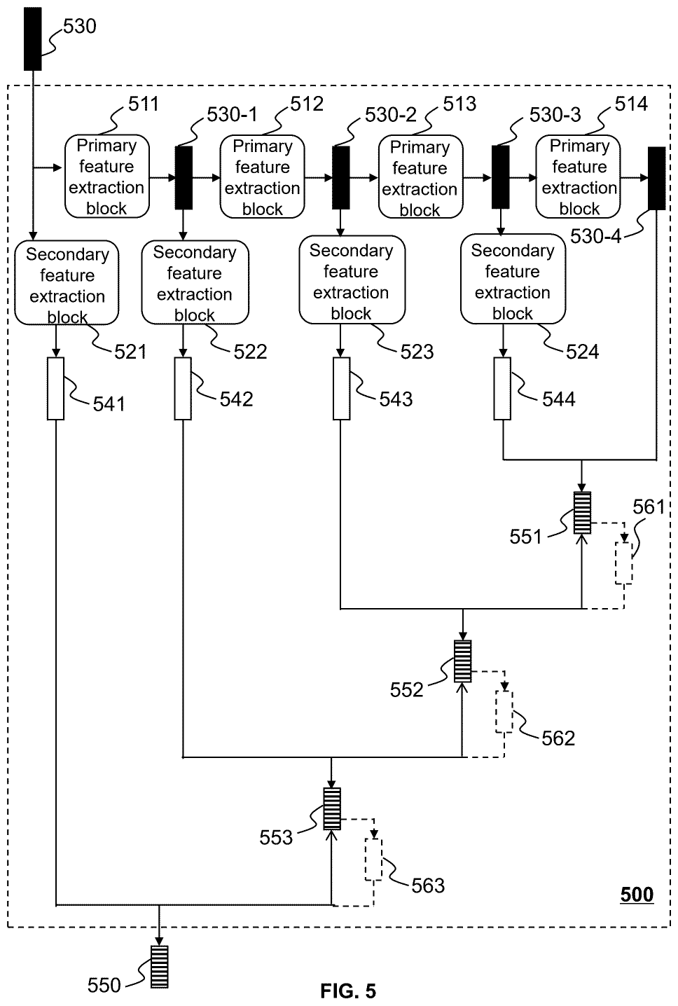

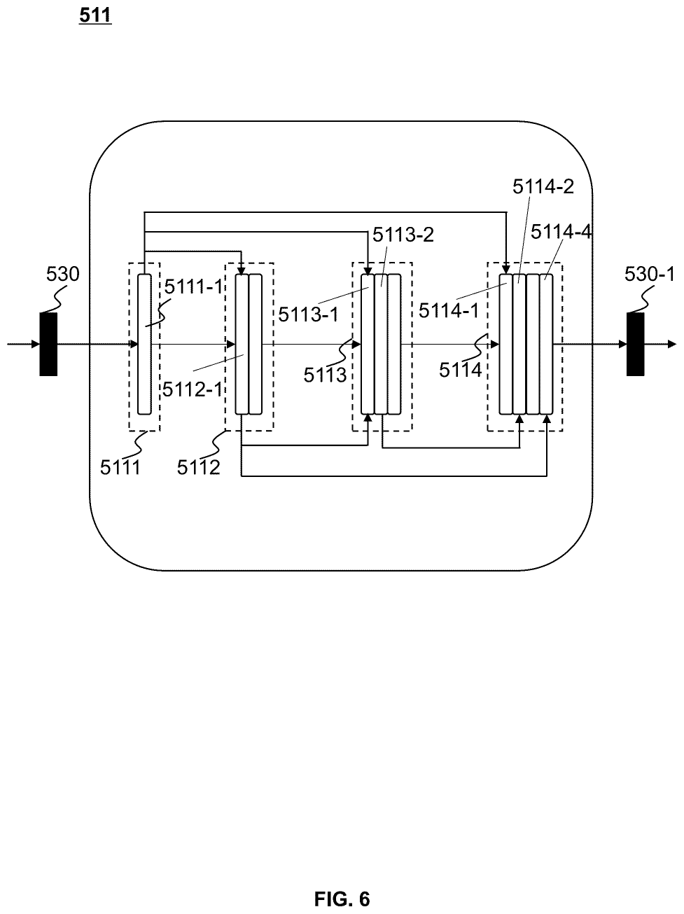

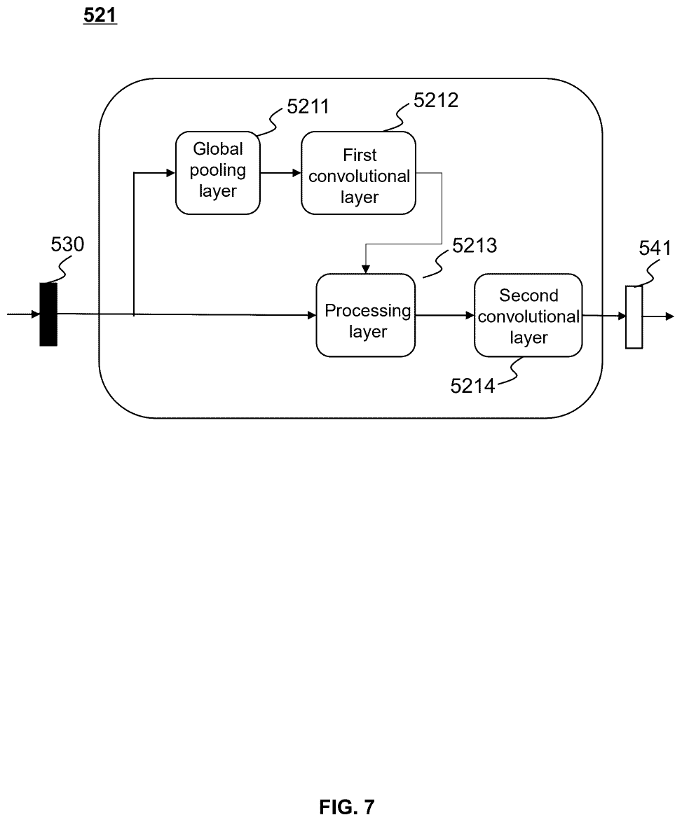

FIG. 5 is a schematic diagram illustrating an exemplary logical structure of a deep learning model according to some embodiments of the present disclosure. FIG. 6 is a schematic diagram illustrating an exemplary logical structure of a primary feature extraction block according to some embodiments of the present disclosure. FIG. 7 is a schematic diagram illustrating an exemplary logical structure of a secondary feature extraction block according to some embodiments of the present disclosure.

In some embodiments, the deep learning model includes a plurality of primary feature extraction blocks that are connected in series and a plurality of secondary feature extraction blocks. For example, as shown in FIG. 5, the deep learning model 500 includes primary feature extraction blocks 511, 512, 513, 514 that are sequentially connected, and secondary feature extraction blocks 521, 522, 523, 524.

The term “sequentially” herein refers to a sequential order along a direction of data flow. It should be understood that FIG. 5 takes only 4 primary feature extraction blocks and 4 secondary feature extraction blocks as an example and is not intended to limit the counts of the primary feature extraction blocks and the secondary feature extraction blocks. In some embodiments, a count of the primary feature extraction blocks and a count of the secondary feature extraction blocks may be set according to actual requirements (e.g., the accuracy in removing artifacts, a speed of obtaining the target reconstructed image, etc.).

The primary feature extraction block is configured to extract a primary feature. For example, the primary feature extraction block may be a residual dense block (RDB) for primary feature extraction.

Among the plurality of primary feature extraction blocks included in the deep learning model, an input of a first primary feature extraction block includes the first reconstructed image. An input of each of the primary feature extraction blocks other than the first primary feature extraction block includes an output of a previous primary feature extraction block (i.e., the primary feature extracted by the previous primary feature extraction block, hereinafter referred to as a first intermediate sub-feature map). An output of a last primary feature extraction block of the plurality of primary feature extraction blocks includes a first sub-feature map.

Exemplarily, taking the deep learning model 500 as an example, the input of the first primary feature extraction block (i.e., a primary feature extraction block 511) includes a first reconstructed image 530. The input of each of the primary feature extraction blocks (including primary feature extraction blocks 512, 513, 514) other than the first primary feature extraction block 511 includes the output of the previous primary feature extraction block (i.e., the input of the primary feature extraction block 512 includes a first intermediate sub-feature map 530-1 output by the primary feature extraction block 511, the input of the primary feature extraction block 513 includes a first intermediate sub-feature map 530-2 output by the primary feature extraction block 512, and the input of the primary feature extraction block 514 includes a first intermediate sub-feature map 530-3 output by the primary feature extraction block 513). The output of the last primary feature extraction block (i.e., the primary feature extraction block 514) includes a first sub-feature map 530-4.

In some embodiments, the input of each of the primary feature extraction blocks other than the first primary feature extraction block may also include the first reconstructed image input into the first primary feature extraction block. By inputting the first reconstructed image into each of the primary feature extraction blocks other than the first primary feature extraction block, original image information can be preserved while removing artifacts, avoiding the loss of details due to a plurality of nonlinear transformations in deep networks.

In some embodiments, the primary feature extraction block includes a plurality of primary feature extraction sub-blocks. The plurality of primary feature extraction sub-blocks are sequentially connected to extract the primary feature. Each primary feature extraction sub-block includes at least one convolutional block. In some embodiments, counts of convolutional blocks of the primary feature extraction sub-blocks increase sequentially.

Exemplarily, the primary feature extraction block 511 is taken as an example (other primary feature extraction blocks have the same or similar logical structure as the primary feature extraction block 511). As shown in FIG. 6, the primary feature extraction block 511 includes primary feature extraction sub-blocks 5111, 5112, 5113, and 5114. These four primary feature extraction sub-blocks each include at least one convolutional block, and the count of convolutional blocks within each primary feature extraction sub-block increases sequentially along the direction of data flow. As shown in FIG. 6, the primary feature extraction sub-block 5111 includes 1 convolutional block, the primary feature extraction sub-block 5112 includes 2 convolutional blocks, the primary feature extraction sub-block 5113 includes 3 convolutional blocks, and the primary feature extraction sub-block 5114 includes 4 convolutional blocks.

It should be understood that the 4 primary feature extraction sub-blocks shown in FIG. 6 are not intended to limit the count of the plurality of primary feature extraction sub-blocks. In some embodiments, the count of the plurality of primary feature extraction sub-blocks may be set based on actual needs. Similarly, as shown in FIG. 6, the counts of convolutional blocks in the 4 primary feature extraction sub-blocks start from 1 and increase sequentially with a constant increment of 1. This description serves merely as an example. It should be understood that the count of convolutional blocks in each primary feature extraction sub-block only needs to follow a sequentially increasing pattern. For example, the counts of convolutional blocks in the primary feature extraction sub-blocks could begin with other values (e.g., 2, 3, etc.). As another example, the increment could also adopt other values (e.g., 2, 3, etc.). As still another example, the increment does not need to be constant.

In some embodiments, for each primary feature extraction sub-block, an output of one convolutional block in the primary feature extraction sub-block is input into one convolutional block in each subsequent primary feature extraction sub-block. Exemplarily, as shown in FIG. 6, an output of a convolutional block 5111-1 in the primary feature extraction sub-block 5111 is input into a first convolutional block in each subsequent primary feature extraction sub-block (e.g., a convolutional block 5112-1 in the primary feature extraction sub-block 5112, a convolutional block 5113-1 in the primary feature extraction sub-block 5113, and a convolutional block 5114-1 in the primary feature extraction sub-block 5114). An output of the first convolutional block 5112-1 in the primary feature extraction sub-block 5112 is input into one convolutional block of each subsequent primary feature extraction sub-block (e.g., the first convolutional block 5113-1 in the primary feature extraction sub-block 5113 and the last convolutional block 5114-4 in the primary feature extraction sub-block 5114). An output of the second convolutional block 5113-2 in the primary feature extraction sub-block 5113 is input into one convolutional block in each subsequent primary feature extraction sub-block (e.g., the second convolutional block 5114-2 in the primary feature extraction sub-block 5114).

In some embodiments, each convolutional block includes a convolutional layer, a batch normalization layer, and an activation function layer. The convolutional layer is configured to extract the primary feature from the first reconstructed image or the first intermediate sub-feature map. The batch normalization layer is configured to standardize and batch-normalize the extracted primary feature. The activation function layer is configured to map the standardized and normalized primary feature to convert the standardized and normalized primary feature into nonlinear data.

In some embodiments of the present disclosure, the primary feature extraction block with the above structure has a good effect on extracting the primary feature. By using such primary feature extraction blocks, on the premise of ensuring the performance of the deep learning model, it is realizable to set fewer residual dense blocks in the deep learning model, thereby saving computational resources.

The secondary feature extraction block is configured to extract a secondary feature.

In some embodiments, the secondary feature extraction block may extract the secondary feature based on an attention mechanism.

The primary feature and the secondary feature are only used to distinguish the different results extracted by different feature extraction blocks and do not limit the specific features. It should be understood that “primary feature” and “secondary feature” refer to any feature capable of identifying artifacts, including but not limited to a gray-scale distribution feature, a gradient feature, a texture feature, or the like, or any combination thereof.

In some embodiments, the count of the secondary feature extraction blocks is the same as the count of the primary feature extraction blocks, i.e., each secondary feature extraction block corresponds to one primary feature extraction block. The input of each secondary feature extraction block is the same as the input of the corresponding primary feature extraction block. The output of each secondary feature extraction block is a second sub-feature map. The second sub-feature map includes a feature optimization result. The feature optimization result is optimized feature information, which is crucial for the final removal of artifacts. The feature optimization result includes a result of enhancing artifact features, a result of fusing multi-scale features, or a result of optimizing a feature space. For example, the result of enhancing artifact features includes at least one of results of enhancing feature response in artifact regions, suppressing of interfering features in non-artifact regions, and improving differentiation between the artifact features and normal tissue features via an attentional mechanism. The result of fusing multi-scale features includes at least one of results of combining local detail features and global contextual features, optimizing correlations between features of different scales, and preserving important edge and texture information. The result of optimizing the feature space includes at least one of results of separating artifact features from real tissue features in the feature space or establishing a feature representation more conducive to artifact removal, providing an optimized feature basis for subsequent multi-round computational processing.

Exemplarily, taking the deep learning model 500 as an example, as shown in FIG. 5, secondary feature extraction blocks 521, 522, 523, and 524 correspond to primary feature extraction blocks 511, 512, 513, and 514, respectively. The inputs of the secondary feature extraction blocks 521, 522, 523, and 524 are the same as the inputs of the primary feature extraction blocks 511, 512, 513, and 514, respectively. For example, the input of the secondary feature extraction block 521 is the first reconstructed image 530. The input of the secondary feature extraction block 522 is the first intermediate sub-feature map 530-1 (optionally, further including the first reconstructed image 530). The input of the secondary feature extraction block 523 is the first intermediate sub-feature map 530-2 (optionally, further including the first reconstructed image 530). The input of the secondary feature extraction block 524 is the first intermediate sub-feature map 530-3 (optionally, further including the first reconstructed image 530). The outputs of the secondary feature extraction blocks 521, 522, 523, and 524 are second sub-feature maps 541, 542, 543, and 544, respectively.

In some embodiments, a secondary feature extraction block includes a global pooling layer, a first convolutional layer, a processing layer, and a second convolutional layer. For example, taking the secondary feature extraction block 521 as an example (other secondary feature extraction blocks have the same or similar logical structure as that of the secondary feature extraction block 521), as shown in FIG. 7, the secondary feature extraction block 521 includes a global pooling layer 5211, a first convolutional layer 5212, a processing layer 5213, and a second convolutional layer 5214.

The global pooling layer and the first convolutional layer are configured to extract weights of globally important information in input data (e.g., the first reconstructed image or the first intermediate sub-feature map) of the secondary feature extraction block.

The processing layer is configured to process the input data (e.g., the first reconstructed image or the first intermediate sub-feature map) of the secondary feature extraction block and the weights of the global important information to obtain a processing result. In some embodiments, the aforementioned processing is multiplication.

The second convolutional layer is configured to process the processing result to obtain the second sub-feature map. In some embodiments, the aforementioned processing is a convolution.

In some embodiments of the present disclosure, by determining the second sub-feature map, it helps the deep learning model sample features that are more conducive to removing artifacts from the first reconstructed image.

In some embodiments, the target reconstructed image is determined based on the first sub-feature map and the plurality of second sub-feature maps.

In some embodiments, the processing module 320 or the processing device 140 may generate the target reconstructed image by performing a plurality of iterations (i.e., a count of N) on the first sub-feature map based on the plurality of second sub-feature maps output by the plurality of secondary feature extraction blocks.

In some embodiments, the plurality of iterations include: in a first iteration, generating an intermediate processing result by processing the first sub-feature map and the second sub-feature map output by the last secondary feature extraction block of the plurality of secondary feature extraction blocks; in each subsequent iteration of the plurality of iterations, generating an updated intermediate processing result by sequentially processing, in a reverse order, the second sub-feature map output by a previous secondary feature extraction block and the intermediate processing result generated in a previous iteration, then proceeding to the next iteration; and generating an artifact-optimized reconstructed image, i.e., a target reconstructed image 550, by processing the second sub-feature map output by the first secondary feature extraction block and the intermediate processing result generated in the previous iteration. In some embodiments, the aforementioned processing includes convolution.

In some embodiments, a count of the plurality of secondary feature extraction blocks is N which is an integer greater than 1. The generating the target reconstructed image includes: performing N iterations in a reverse order based on the first sub-feature map and the second sub-feature maps output by the plurality of secondary feature extraction blocks, including: in a first iteration of the N iterations, generating an intermediate processing result by processing the first sub-feature map and the second sub-feature map output by a last secondary feature extraction block of the plurality of secondary feature extraction blocks; and in an ith iteration of the N iterations, generating an intermediate processing result based on the second sub-feature map output by an [N−(i−1)]th secondary feature extraction block of the plurality of secondary feature extraction blocks and the intermediate processing result generated in an (i−1)th iteration, wherein i≠1; and determining the intermediate processing result generated in a last iteration of the N iterations as the target reconstructed image. In some embodiments, in each iteration, the intermediate processing result is generated by performing a convolution on the first sub-feature map and the corresponding second sub-feature map.

Still taking the deep learning model 500 as an example and referring to FIG. 5, exemplary iterations may include: in a first iteration, generating an intermediate processing result 551 by processing the first sub-feature map 530-4 and the second sub-feature map 544 output by a last secondary feature extraction block (i.e., the secondary feature extraction block 524); in a second iteration, generating an intermediate processing result 552 by processing the intermediate processing result 551 and the second sub-feature map 543 output by the secondary feature extraction block 523; in a third iteration, generating an intermediate processing result 553 by processing the intermediate processing result 552 and the second sub-feature map 542 output by the secondary feature extraction block 522; and in a fourth (i.e., last) iteration, generating the target reconstructed image 550 by processing the intermediate processing result 553 (i.e., the intermediate processing result obtained from the previous (i.e. third) iteration) and the second sub-feature map 541 output by the secondary feature extraction block 521 (i.e., the first secondary feature extraction block). In some embodiments, the processing in each iteration includes convolution.

In some embodiments, each iteration in the above embodiments further includes upsampling. For example, the upsampling includes deconvolution, transposed convolution, or interpolation techniques (e.g., nearest-neighbor interpolation, bilinear interpolation, etc.).

Continuing the previous example, still taking the deep learning model 500 as an example and referring to FIG. 5, exemplary iterations may include: in a first iteration, generating the intermediate processing result 551 by processing the first sub-feature map 530-4 and the second sub-feature map 544 output by a last secondary feature extraction block (i.e., the secondary feature extraction block 524), then upsampling the intermediate processing result 551 to generate an upsampled intermediate processing result 561; in a second iteration, generating the intermediate processing result 552 by processing the upsampled intermediate processing result 561 and the second sub-feature map 543 output by the secondary feature extraction block 523, then upsampling the intermediate processing result 552 to generate an upsampled intermediate result 562; in a third iteration, generating the intermediate processing result 553 by processing the upsampled intermediate result 562 and the second sub-feature map 542 output by the secondary feature extraction block 522, then upsampling the intermediate processing result 553 to generate an upsampled intermediate result 563; and in a fourth (i.e., last) iteration, generating an artifact-optimized reconstructed image (i.e., the target reconstructed image) 550 by processing the upsampled intermediate result 563 (i.e., the upsampled intermediate result obtained from the previous (i.e. third) iteration) and the second sub-feature map 541 output by the secondary feature extraction block 521 (i.e., the first secondary feature extraction block). In some embodiments, the processing in each iteration includes convolution.

In some embodiments of the present disclosure, the above multi-iteration process makes the extracted features more optimized and improves the artifact removal effect on the first reconstructed image. By introducing upsampling, the resolution of the feature maps can be increased, further improving the artifact removal effect.

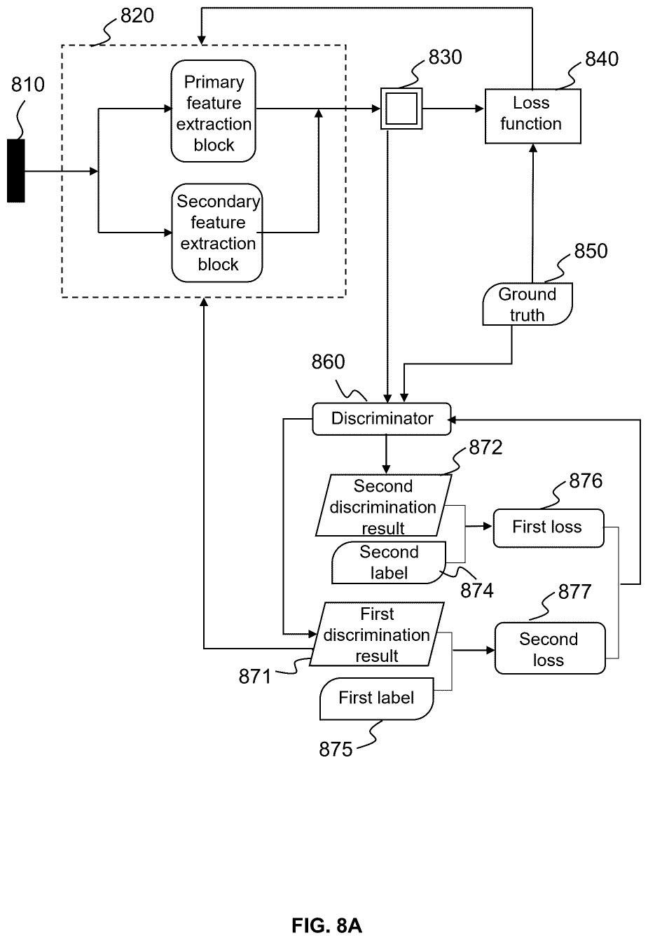

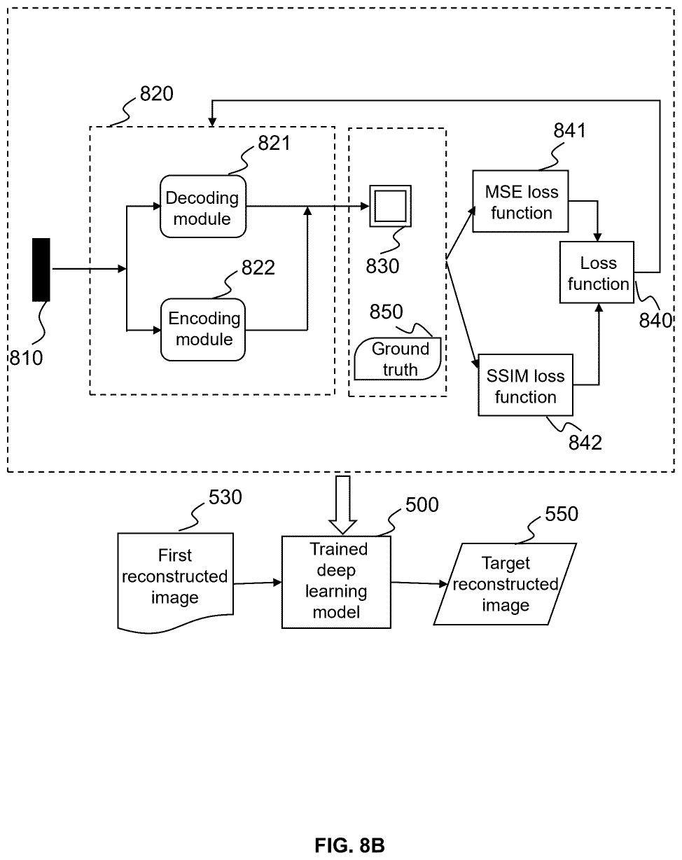

FIG. 8A is a schematic diagram illustrating an exemplary process for training a deep learning model according to some embodiments of the present disclosure. FIG. 8B is a schematic diagram illustrating an exemplary process for training a deep learning model according to some embodiments of the present disclosure.

In some embodiments, the trained deep learning model is obtained by performing a training process. The training process includes: obtaining a plurality of training samples; and performing a plurality of iterations based on the plurality of training samples. The training sample used for training the deep learning model may include a sample first reconstructed image and a sample target reconstructed image. An image quality of the sample first reconstructed image is lower than that of the sample target reconstructed image. The sample first reconstructed image serves as an model input of the training process, and the sample target reconstructed image serves as a label of the sample first reconstructed image. The training samples may be determined based on historical reconstruction data. For example, a historical reconstructed image including artifacts (e.g., motion artifacts, metal artifacts, limited-angle artifacts, etc.) may be served as a sample first reconstructed image, and an artifact-optimized reconstructed image obtained by, e.g., performing an artifact-optimized algorithm on the historical reconstructed image may be served as a sample target reconstructed image. As another example, an artifact-free reconstructed image (e.g., a CT image reconstructed using full-angle projection data) may be served as a sample target reconstructed image. A low-quality image generated by adding noise or other degradation to the sample target reconstructed image may be served as a sample first reconstructed image. As a further example, full-angle projection data may be obtained. An image generated by reconstructing the full-angle projection data (e.g., using filtered back projection (FBP)) may be served as a sample target reconstructed image. The full-angle projection data may be processed to simulate limited-angle projection data, e.g., removing projection data corresponding to a portion of projection angles from the full-angle projection data. An image generated by reconstructing the limited-angle projection data (e.g., using filtered back projection (FBP)) may be served as a sample first reconstructed image.

Referring to FIG. 8A, each of the plurality of iterations includes: inputting a sample first reconstructed image 810 into an initial deep learning model 820 to obtain an intermediate output result 830 output by the initial deep learning model 820; determining a value of a loss function 840 based on the intermediate output result 830 and a ground truth (i.e., the sample target reconstructed image) 850; iteratively updating parameters of the initial deep learning model 820 based on the value of the loss function 840 to obtain an intermediate deep learning model; determining whether the current training process satisfies a preset condition; under a condition that the current training process has two situations including satisfying the preset condition and not satisfying the preset condition, in response to determining that the current iteration satisfies the preset condition, determining the intermediate deep learning model obtained in the current iteration as the trained deep learning model (e.g., the deep learning model 500). The preset condition includes at least convergence of the loss function or a count of the plurality of iterations reaches a count threshold. In response to determining that the current iteration does not satisfy the preset condition, the processing device 140 initiates a new iteration. In some embodiments, the structure of the initial deep learning model 820 is similar to that of the deep learning model 500, but with different parameters. After the sample first reconstructed image 810 is input into the initial deep learning model 820, the initial deep learning model 820 processes the sample first reconstructed image 810 through the primary and secondary feature extraction blocks and outputs the intermediate output result 830.

In some embodiments, each of the plurality of iterations further includes: processing the intermediate output result output by the initial deep learning model using a discriminator to obtain a first discrimination result; and iteratively updating the parameters of the initial deep learning model based on the value of the loss function and the first discrimination result.

The first discrimination result represents a probability, considered by the discriminator, that the intermediate output result is a real image rather than an image generated by a generator (e.g., the initial deep learning model), e.g., a similarity probability between the intermediate output result and the ground truth.

In some embodiments, as shown in FIG. 8A, the medical imaging system 300 or the processing device 140 may input the intermediate output result 830 into the discriminator 860, use the discriminator 860 to output a similarity probability (i.e., a first discrimination result 871) between the intermediate output result 830 and the ground truth 850, determine a total loss based on the value of loss function 840 and the first discrimination result 871 (e.g., determining a weighted sum of the value of the loss function 840 and the first discrimination result 871 as the total loss), and backpropagate to iteratively update the parameters of the initial deep learning model based on the total loss.

In some embodiments, during the training process of the initial deep learning model 820, the medical imaging system 300 or the processing device 140 may also optimize the parameters of the discriminator 860. In some embodiments, the medical imaging system 300 or the processing device 140 may input the sample target reconstructed image (i.e., the ground truth) 850 into the discriminator 860 and set a label (a second label 874) of the sample target reconstructed image as 1, defining that the sample target reconstructed image is a real image rather than an image generated by a generator (e.g., the initial deep learning model). The discriminator 860 outputs a second discrimination result 872 indicating a probability, considered by the discriminator 860, that the sample target reconstructed image is a real image rather than an image generated by a generator (e.g., the initial deep learning model), e.g., a similarity probability to the sample target reconstructed image. The processing device 140 may set a label (a first label 875) of the intermediate output result 830 as 0, defining that the intermediate output result 830 is an image generated by a generator (e.g., the initial deep learning model). The processing device 140 may determine a first loss 876 by determining a difference between the second discrimination result 872 and the second label 874. The processing device 140 may determine a second loss 877 by determining a difference between the first discrimination result 871 and the first label 875. The processing device 140 may iteratively update the parameters of the discriminator 860 based on the first loss and the second loss.

In some embodiments, before training the deep learning model, the sample first reconstructed image may be normalized.

Exemplarily, each pixle of the sample first reconstructed image may be normalized based on the following Equation (1):

$${I}^{′} = \frac{I - μ}{σ} , \qquad ( 1 )$$

where I′ denotes a pixel value of a pixel in the sample first reconstructed image after normalization, I denotes a pixel value of the pixel before normalization, μ denotes an average of pixel values of all pixels in the sample first reconstructed image before normalization, and σ denotes a standard deviation of the pixel values of all pixels in the sample first reconstructed image before normalization.

In some embodiments, the sample target reconstructed image may also be normalized before training the deep learning model. The manner of normalizing the sample target reconstructed image is similar to the manner of normalizing the sample first reconstructed image.

In some embodiments, before inputting the first reconstructed image into the trained deep learning model 500, the processing device 140 may also normalize the first reconstructed image using a manner similar to the one described for the sample first reconstructed image.

In some embodiments, the loss function 840 includes a mean squared error (MSE) loss function.

In some embodiments, the loss function 840 includes a structural similarity (SSIM) loss function.

In some embodiments, the loss function 840 may include only one of the MSE loss function or the SSIM loss function. In some embodiments, in order to optimize a training evaluation system of the deep learning model and further improve the performance of the trained deep learning model, the loss function 840 may include both the MSE loss function and the SSIM loss function. In some embodiments, when the loss function 840 includes both the MSE loss function and the SSIM loss function, the deep learning model may also be further improved by dynamically determining a different weight for each loss function, thereby improving the accuracy of artifact removal.

Exemplarily, as shown in FIG. 8B, the initial deep learning model 820 includes a decoding module 821 and an encoding module 822. The decoding module 821 corresponds to a plurality of sequentially connected initial primary feature extraction blocks for extracting a primary feature. The encoding module 822 is combined with an attention mechanism extraction and corresponds to a plurality of initial secondary feature extraction blocks for extracting a secondary feature. The loss function 840 includes an MSE loss function 841 and an SSIM loss function 842. An exemplary training process for the deep learning model may include: in each of the plurality of iterations, determining an output of the decoding module 821 (i.e., the plurality of initial primary feature extraction blocks) and an output of the encoding module 822 (i.e., extracted by the plurality of initial secondary feature extraction blocks through the attention mechanism) by inputting the sample first reconstructed image 810 into the decoding module 821 and the encoding module 822 of the initial deep learning model 820; determining (e.g., via multiplicative calculation) the intermediate output result 830 based on the output of the decoding module 821 and the output of the encoding module 822; determining a value of the MSE loss function 841 based on the intermediate output result 830 and the ground truth 850; determining a value of the SSIM loss function 842 based on the intermediate output result 830 and the ground truth 850; iteratively updating the parameters of the initial deep learning model 820 based on the values of the MSE loss function and the SSIM loss function to obtain an intermediate deep learning model; determining whether the current training process satisfies a preset condition; under a condition that the current training process has two situations including satisfying the preset condition and not satisfying the preset condition, in response to determining that the current iteration satisfies the preset condition, determining the intermediate deep learning model obtained in the current iteration as the trained deep learning model (e.g., the deep learning model 500); and in response to determining that the current iteration does not satisfy the preset condition, the processing device 140 initiates a new iteration. The first reconstructed image may be input into the trained deep learning model to obtain the artifact-optimized reconstructed image (i.e., the target reconstructed image). More descriptions regarding the first reconstructed image and the target reconstructed image may be found in FIGS. 4 to 15, and the descriptions thereof.

In some embodiments, a difference between the intermediate output result and the ground truth is directly calculated at the pixel-level based on the MSE loss function. Equation of the MSE loss function is expressed as Equation (2) below:

$$M S E = \frac{1}{M} {\sum }_{i = 1}^{M} {( {O}_{i} - {T}_{i} )}^{2} , \qquad ( 2 )$$

where MSE denotes a value of the MSE loss function, Oᵢ denotes a pixel value of an ith pixel in the intermediate output result, Tᵢ denotes a pixel value of a pixel at the same position as the ith pixel in the ground truth, and M denotes a total count of pixels in the intermediate output result. MSE loss ensures that an output image of the trained deep learning model matches the ground truth in pixel grayscale, reflecting absolute differences in overall brightness, contrast, etc., between the intermediate output result and the ground truth.

In some embodiments, the SSIM loss function focuses primarily on structural information of images, not just pixel-level differences. The SSIM loss function gives a similarity score between the intermediate output result and the ground truth by measuring similarity in terms of luminance, contrast, and structure. SSIM loss encourages the trained deep learning model not only to match pixel values but also to better recover textures, edges, and other structural information, thereby enhancing visual quality and realism. By determining the value of the MSE loss function, the difference between the intermediate output result and the ground truth at each pixel can be evaluated. By determining the value of the SSIM loss function, a structural similarity between the intermediate output result and the ground truth can be evaluated locally and globally. By weighting and summing the value of the MSE loss function and the value of the SSIM loss function, and using the weighted sum as the value of the loss function 840, a gradient during backpropagation comes from both pixel-level and structural-level differences, thereby enabling the trained deep learning model to remove artifacts while maintaining the realism of details and overall structure.

In some embodiments of the present disclosure, the above training process enables the trained deep learning model to more accurately determine an artifact-optimized reconstructed image. By introducing different loss functions during training, the training evaluation system is optimized, further enhancing the performance of the trained deep learning model. By normalizing the sample first reconstructed images in the training samples before training, it helps the loss function converge, improving the accuracy of the trained deep learning model.

FIG. 9A is a block diagram of an exemplary medical imaging system according to some embodiments of the present disclosure. In some embodiments, as shown in FIG. 9A, a medical imaging system 900-1 includes a scanning acquisition module 901, a data reconstruction module 902, an interpolation module 903, and an image reconstruction module 904. The medical imaging system 900-1 may be implemented on the processing device 140 through hardware or software.

The scanning acquisition module 901 is configured to obtain a plurality of sets of initial scanning data. Each of the plurality of sets of initial scanning data is acquired by performing a circle of full-angle scanning on a target subject using an imaging device (e.g., the imaging device 110).

The data reconstruction module 902 is configured to, for each of the plurality of sets of initial scanning data, generate initial reconstruction data by performing reconstruction based on the set of initial scanning data.

The interpolation module 903 is configured to generate a third reconstructed image corresponding to a target time point by performing, in a chronological order, interpolating based on the initial reconstruction data of the plurality of sets of initial scanning data.

The image reconstruction module 904 is configured to generate the first reconstructed image based on the third reconstructed image and a portion of the plurality of sets of initial scanning data.

More descriptions may be found in FIGS. 9B-11 and their related descriptions.

It should be noted that the above description of the medical imaging system 900-1 and its modules is for convenience of description only and should not limit the present disclosure to the scope of the illustrated embodiments. It is understood that for those skilled in the art, after understanding the principles of the system, they may combine the modules arbitrarily or form subsystems connected to other modules without departing from these principles. In some embodiments, the scanning acquisition module 901, the data reconstruction module 902, the interpolation module 903, and the image reconstruction module 904 disclosed in FIG. 9A may be different modules within one system, or one module may implement the functions of two or more of the above modules. For example, the modules may share a storage module, or the modules may each have their own respective storage modules. Such variations are all within the scope of the present disclosure.

FIG. 9B is a flowchart illustrating an exemplary process for obtaining a first reconstructed image according to some embodiments of the present disclosure. As shown in FIG. 9B, process 900-2 includes the following operations. In some embodiments, process 900-2 may be performed by the medical imaging system 900-1 or the processing device 140.

In 910, the processing device 140 obtains a plurality of sets of initial scanning data. Each of the plurality of sets of initial scanning data is acquired by performing a circle of full-angle scanning on a target subject using an imaging device (e.g., the imaging device 110).

In some embodiments, one circle of full-angle scanning may include a full scanning or a short scanning. In the full scanning, a radiation source rotates 360° around the target subject, and initial scanning data is collected from projection angles of 360° (e.g., acquiring the initial scanning data at a preset angle interval, e.g., 0.1°, 0.5°, 1°, etc.). In the short scanning, the radiation source rotates within an angle range less than 360° (e.g., 180°+a fan angle or cone angle of a radiation beam emitted from the radiation source), and initial scanning data is collected from the angle range less than 360° (e.g., acquiring the initial scanning data at a preset angle interval, e.g., 0.1°, 0.5°, 1°, etc.).

In some embodiments, the medical imaging system 900-1 or the processing device 140 may control the imaging device to perform a plurality of circles of full-angle scanning on the target subject to obtain the plurality of sets of initial scanning data.

In some embodiments, a rotation mode of the imaging device includes alternating rotation (e.g., covering 0° to 200° clockwise and then returning counterclockwise) and rotation in one fixed direction (e.g., always performing a clockwise rotation or always performing an anticlockwise rotation).

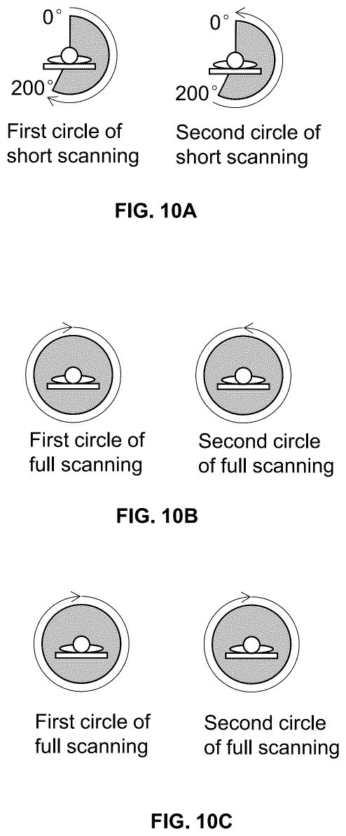

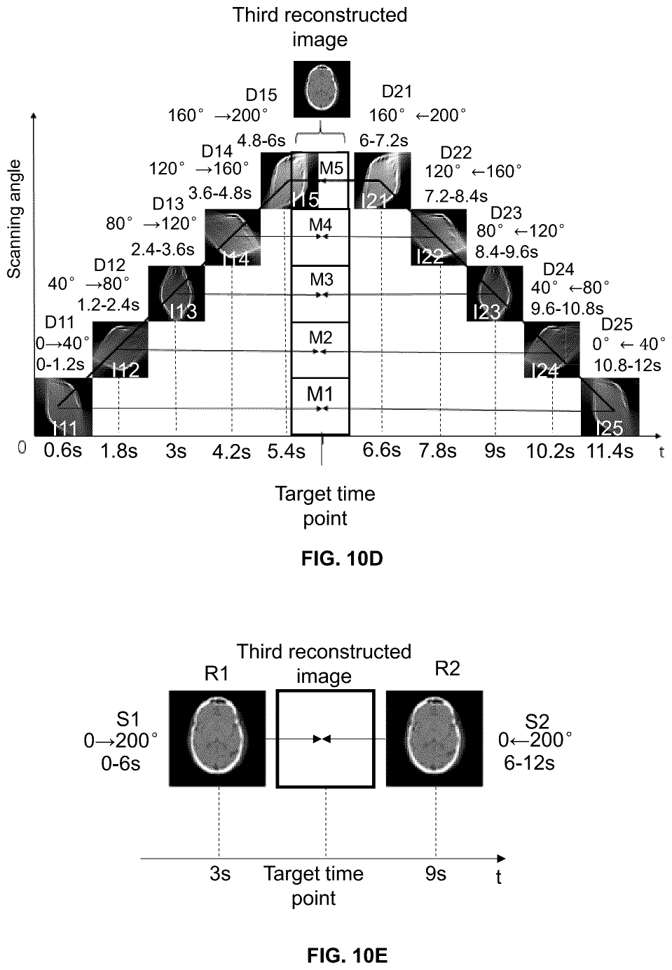

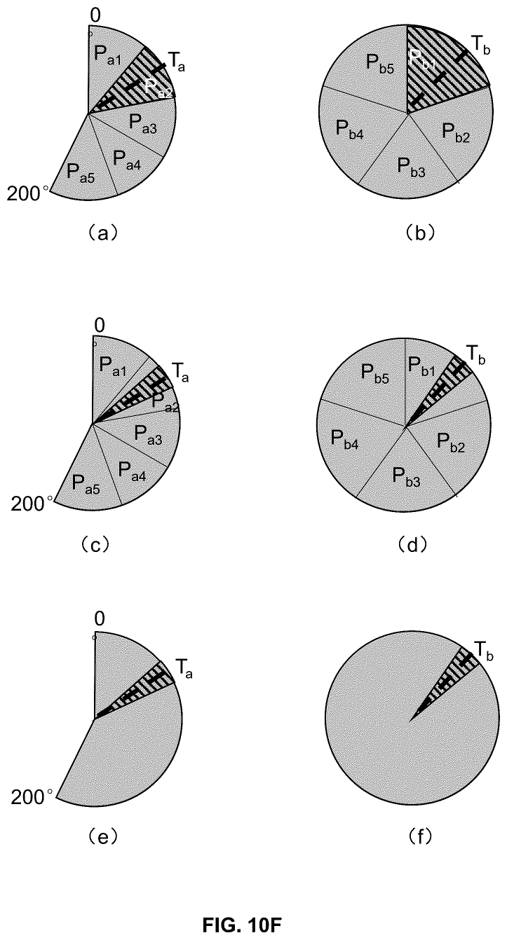

For example, as shown in FIG. 10A, the imaging device performs alternating rotation for a plurality of circles of short scanning of the target subject to obtain the plurality of sets of initial scanning data. For example, a scanning angle when the radiation source of the imaging device is at the 12 o'clock position is defined as 0°. In a first circle, the imaging device rotates clockwise from the 0° position to the 200° position; and in a second circle, the imaging device rotates counterclockwise from the 200° position back to the 0° position, and so on. It should be noted that other position (e.g., 9 o'clock position) of the radiation source can also be designated as the 0° position, which is not limited herein.

As another example, as shown in FIG. 10B, the imaging device performs alternating rotation for a plurality of circles of full scanning of the target subject to obtain the plurality of sets of initial scanning data. In a first circle, the imaging device rotates 360° clockwise from 0° back to 0°; and in a second circle, the imaging device rotates 360° counterclockwise from 0° back to 0°, and so on.

As a further example, as shown in FIG. 10C, the imaging device rotates in one direction and performs a plurality of circles of full scanning on the target subject to obtain the plurality of sets of initial scanning data. In a first circle, the imaging device rotates 360° clockwise from 0° back to 0°; and in a second circle, the imaging device continues rotating 360° clockwise from 0° back to 0°, and so on.

In some embodiments, a count of the plurality of sets of initial scanning data may be determined based on actual requirements. Taking brain perfusion CT imaging as an example, after intravenous injection of a contrast agent, the time for the contrast agent to flow into the brain and out of the brain is about 30-40 seconds. If the time for the imaging device to perform one circle of full-angle scanning is fixed, a count of circles of full-angle scanning needed may be determined based on the time for the contrast agent to flow into the brain and out of the brain and the time for the imaging device to perform one circle of full-angle scanning. For instance, if the time for the imaging device to perform one circle of full-angle scanning is 6 s, typically 8 circles of full-angle scanning may be performed, and 8 sets of initial scanning data may be obtained.

Rays emitted by the radiation source pass through the target subject and are received by the detector. These rays are first converted into visible light (e.g., using scintillation crystals to precisely capture ray photons and produce fluorescence), the light signal is converted into an electrical signal (e.g., by a photodiode), then the electrical signal is converted into a digital signal (e.g., by an analog-to-digital converter), and finally the digital signal is input into a computer (e.g., the processing device 140). The digital signal may be called projection data or initial scanning data.

The initial scanning data may be transmitted in real-time to the medical imaging system 900-1 or the processing device 140 after a plurality of circles of full-angle scanning by the imaging device. Alternatively, the imaging device may store the initial scanning data acquired after the plurality of circles of full-angle scanning by the imaging device in a storage component (e.g., the storage device 250) corresponding the imaging device, and the medical imaging system 900-1 or the processing device 140 may retrieve the initial scanning data when needed. The initial scanning data may also be acquired by the medical imaging system 900-1 or the processing device 140 from the imaging device's post-processing workstation or from servers such as picture archiving and communication systems (PACS). This embodiment does not limit the specific manner of acquiring the initial scanning data, as long as the function can be achieved.

In some embodiments, the plurality of sets of initial scanning data correspond to the same scanning angle range. For example, in the full scanning, the plurality of sets of initial scanning data correspond to a scanning angle range of 0°-360°. As another example, in the short scanning, the plurality of sets of initial scanning data correspond to a scanning angle range between 0° and 180°+a fan angle or cone angle of a radiation beam emitted from the radiation source, e.g., 0°-200°.

In 920, for each of the plurality of sets of initial scanning data, the processing device 140 generates initial reconstruction data by performing reconstruction based on the set of initial scanning data.

The initial reconstruction data corresponding to a set of initial scanning data is reconstructed data obtained after reconstructing the set of initial scanning data.

In some embodiments, the medical imaging system 900-1 or the processing device 140 reconstructs each set of initial scanning data separately to obtain the initial reconstruction data corresponding to each set of initial scanning data, thus obtaining a plurality of sets of initial reconstruction data. A count of the plurality of sets of initial reconstruction data is the same as a count of circles of full-angle scanning and a count of the plurality of sets of initial scanning data.

For example, the medical imaging system 900-1 or the processing device 140 may perform filtered back projection (FBP) on each set of initial scanning data to obtain the initial reconstruction data corresponding to the set of initial scanning data. For example, since the short scanning collects incomplete data (not from 360°), the reconstruction process for obtaining the initial reconstruction data requires special algorithms (e.g., weighted filtered back projection) to compensate for data incompleteness and mathematical unsuitability during the reconstruction process. This embodiment does not limit the specific reconstruction manner as long as the function can be achieved.

In some embodiments, for each of the plurality of sets of initial scanning data, the generating initial reconstruction data by performing reconstruction based on the set of initial scanning data includes the following operations. For each set of initial scanning data, the medical imaging system 900-1 or the processing device 140 may determine a plurality of sets of partitioned scanning data by partitioning the set of initial scanning data. Each set of partitioned scanning data corresponds to a scanning region and/or a scanning angle range. For each set of partitioned scanning data, a second reconstructed image is reconstructed. For example, the processing device 140 may perform filtered back projection on each set of partitioned scanning data to obtain the corresponding second reconstructed image. This embodiment does not limit the specific reconstruction manner, as long as the function can be achieved.

In some embodiments, the medical imaging system 900-1 or the processing device 140 may determine the plurality of sets of partitioned scanning data by partitioning each set of initial scanning data into a same count of sets of partitioned scanning data. For example, the medical imaging system 900-1 or the processing device 140 may partition each set of initial scanning data into a preset count of sets of partitioned scanning data. This embodiment does not limit the preset count, as long as the function can be achieved. Specifically, for perfusion scanning, the preset count needs to meet a perfusion temporal resolution requirement.

In some embodiments, the medical imaging system 900-1 or the processing device 140 may determine the preset count using a trained first model. The first model may be a machine learning model. In some embodiments, the first model may include a recurrent neural network (RNN) model, a deep neural network (DNN) model, a convolutional neural network (CNN) model, or the like, or any combination thereof.

In some embodiments, the medical imaging system 900-1 or the processing device 140 inputs the plurality of sets of initial scanning data into the first model, and the first model outputs the preset count. In some embodiments, the input of the first model may further include at least one of imaging information, an imaging portion, or contrast agent information.

The imaging information may include one or more scanning parameters (e.g., a radiation dose, a tube current, a tube voltage, a rotation speed, a scanning angle range, an imaging time, etc.).

The contrast agent information may include at least one the type, concentration, injection dose, or injection speed of the contrast agent.

In some embodiments, the first model is trained using a plurality of first training samples with first label data. In some embodiments, the first training sample may include a plurality of sets of sample initial scanning data. In other embodiments, the first training sample may include a plurality of sets of sample initial scanning data, sample imaging information, a sample imaging portion, sample contrast agent information, etc. The first label data corresponding to the first training sample includes a partition count used to partition the plurality of sets of sample initial scanning data. The first training samples may be determined based on historical scanning data.

In some embodiments, for each first training sample, the medical imaging system 900-1 or the processing device 140 may obtain a plurality of candidate partition counts, determine a first feature for each candidate partition count, determine a first score for each candidate partition count based on the first feature, and select a candidate partition count with the highest score as the first label data for the first training sample. The first feature may include at least one of the image quality of the reconstructed partitioned data obtained after back projection reconstruction performed on each set of partitioned scanning data, the image quality of a third reconstructed image corresponding to a target time point, the image quality of a first reconstructed image, the image quality of a target reconstructed image, a reconstruction speed for obtaining the reconstructed partitioned data, the third reconstructed image, and/or the first reconstructed image, or a computational load for obtaining the reconstructed partitioned data, the third reconstructed image, and/or the first reconstructed image. For more details about the third target reconstructed image corresponding to the target time point, the first target reconstructed image, and the target reconstructed image, please refer to the following description. The higher the image quality of the reconstructed partitioned data, the higher the image quality of the third reconstructed image, the higher the image quality of the first reconstructed image, the higher the image quality of the target reconstructed image, the faster the reconstruction speed, and the smaller the computational load, the higher the first score.

In some embodiments, the medical imaging system 900-1 or the processing device 140 may execute the following training process to obtain the first model. The training process includes: obtaining a plurality of first training samples with first label data to form a first training sample set, and performing a plurality of iterations based on the first training sample set. At least one iteration includes: selecting one or more first training samples from the first training sample set; inputting the one or more first training samples into an initial first model to obtain a model prediction output corresponding to the one or more first training samples; substituting the model prediction output and the first label data corresponding to the one or more first training samples into a predefined loss function to determine a value of the loss function; iteratively updating model parameters of the initial first model based on the value of the loss function; in response to determining that a termination condition is met, terminating the iterations to obtain the trained first model. The iteratively updating model parameters of the initial first model may be carried out in various manners. For example, the iterative updating is carried out based on a gradient descent technique. The termination condition may include convergence of the loss function or reaching a maximum iteration count.

In some embodiments of the present disclosure, by using a machine learning model to determine the preset count for partition, the self-learning capability of the machine learning model can be utilized to find patterns from a large amount of historical data, and the correlation relationship between the sample initial scanning data and the partition count can be obtained, thus improving the accuracy and efficiency of determining the preset count for partition, thereby ensuring the reconstruction speed.

In some embodiments, after obtaining the plurality of sets of initial scanning data acquired by performing a plurality of circles of full-angle scanning, the medical imaging system 900-1 or the processing device 140 may determine different scanning regions corresponding to the plurality of sets of initial scanning data, and partition each set of initial scanning data based on the scanning regions. That is to say, if a portion of a set of initial scanning data of a first circle corresponding to a first scanning region is determined as a set of partitioned scanning data corresponding to the first scanning region, a portion of a set of initial scanning data of a second circle corresponding to the first scanning region is also required to be determined as a set of partitioned scanning data corresponding to the first scanning region, and so on. A set of initial scanning data may be partitioned into a plurality of sets of partitioned scanning data corresponding to multiple different scanning regions. The multiple scanning regions corresponding to each set of initial scanning data are the same.

Assuming the preset count is 3, i.e., each set of initial scanning data is partitioned into 3 sets of partitioned scanning data. A set of initial scanning data of a first circle is partitioned into a first set of partitioned scanning data corresponding to a first scanning region, a second set of partitioned scanning data corresponding to a second scanning region, and a third set of partitioned scanning data corresponding to a third scanning region. A set of initial scanning data of a second circle is partitioned into a fourth set of partitioned scanning data corresponding to the first scanning region, a fifth set of partitioned scanning data corresponding to the second scanning region, and a sixth set of partitioned scanning data corresponding to the third scanning region. That is to say, the scanning region corresponding to the first set of partitioned scanning data in the set of initial scanning data of the first circle is the same as the scanning region corresponding to the fourth set of partitioned scanning data in the set of initial scanning data of the second circle, the scanning region corresponding to the second set of partitioned scanning data in the set of initial scanning data of the first circle is the same as the scanning region corresponding to the fifth set of partitioned scanning data in the set of initial scanning data of the second circle, and the scanning region corresponding to the third set of partitioned scanning data in the set of initial scanning data of the first circle is the same as the scanning region corresponding to the sixth set of partitioned scanning data in the set of initial scanning data of the second circle.

As shown in FIG. 10D, each of two sets of initial scanning data (e.g., a set of initial scanning data of a first circle and a set of initial scanning data of a second circle) is partitioned into 5 sets of partitioned scanning data, resulting in 10 sets of partitioned scanning data and 10 corresponding second reconstructed images, wherein second reconstructed image I11 of the first circle corresponds to the same anatomical position information as the second reconstructed image I25 of the second circle, the second reconstructed image I12 of the first circle corresponds to the same anatomical position information as the second reconstructed image I24 of the second circle, the second reconstructed image I13 of the first circle corresponds to the same anatomical position information as the second reconstructed image I23 of the second circle, the second reconstructed image I14 of the first circle corresponds to the same anatomical position information as the second reconstructed image I22 of the second circle, and the second reconstructed image I15 of the first circle corresponds to the anatomical position information as the second reconstructed image I21 of the second circle.

In some embodiments, within the set of initial scanning data of the same circle, the scanning angle ranges and scanning time ranges corresponding to the plurality of sets of partitioned scanning data may be different, and the different scanning angle ranges or scanning time ranges are contiguous (e.g., non-overlapping and gapless in coverage). For example, a set of initial scanning data is partitioned into a first set of partitioned scanning data and a second set of partitioned scanning data. The first set of partitioned scanning data corresponds to a first scanning angle range and a first scanning time range, and the second set of partitioned scanning data corresponds to a second scanning angle range and a second scanning time range. The first scanning angle range and the second scanning angle range are contiguous, and the first scanning time range and the second scanning time range are contiguous.

In some embodiments of the present disclosure, two ways of partitioning each set of initial scanning data are provided, one is to partition each set of initial scanning data according to the same count, and the other is to partition each set of initial scanning data according to the same scanning region, so that the user can choose by himself according to the actual needs, improving the applicability of the image reconstruction method.

The second reconstructed image is a reconstructed image obtained by reconstructing a set of partitioned scanning data. Each set of partitioned scanning data corresponds to one second reconstructed image.

In some embodiments, each set of partitioned scanning data corresponds to a scanning angle range, a scanning time range, and a scanning region of the target subject. The scanning angle ranges and the scanning time ranges of a plurality of sets of partitioned scanning data of one set of initial scanning data are different and contiguous (e.g., non-overlapping and gapless in coverage). The counts of the sets of partitioned scanning data of the plurality of sets of initial scanning data are the same. The multiple scanning angle ranges and/or the multiple scanning regions of each set of initial scanning data are the same. That is, for one set of partitioned scanning data D1 in one set of initial scanning data, it is possible to find a set of partitioned scanning data D2 in another set of initial scanning data that has the same scanning angle range and/or the same scanning region as D1, but has a different scanning time range from D1. The spans of the scanning angle ranges of all sets of partitioned scanning data are the same, and the spans of the scanning time ranges of all sets of partitioned scanning data are the same.

For example, referring to FIG. 10A and FIG. 10D, the imaging device 110 performs two circles of short scanning through alternating rotation to obtain two sets of initial scanning data. A scanning angle range corresponding to each circle of short scanning is 0°-200°, and a span of scanning time corresponding to each circle of scanning is 6 s. In a first circle, the imaging device obtains a first set of initial scanning data S1 by rotating clockwise from 0° position to 200° position, with a scanning time range of 0-6 s. In a second circle, the imaging device obtain a second set of initial scanning data S2 by rotating counterclockwise from the 200° position to the 0° position, with a scanning time range of 6-12 s. It should be noted that scanning between two adjacent circles of full-angle scanning may not be temporally continuous, a typical interval of approximately 1 s may exist. The assumption of temporal continuity here is solely for illustrative purposes.

As shown in FIG. 10D, the horizontal axis in FIG. 10 represents scanning time, the vertical axis in FIG. 10 represents scanning angle. The processing device 140 partitions S1 into 5 sets of partitioned scanning data D11-D15, and S2 into 5 sets of partitioned scanning data D21-D25. The processing device 140 reconstructs D11-D15 to genterate second reconstructed images I11-I15, and reconstructs D21-D25 to genterate second reconstructed images I21-I25.

As shown in FIG. 10D, a span of the scanning angle range of each set of partitioned scanning data is 40°, and a span of the scanning time range of each set of partitioned scanning data is 1.2 s. For example, D11 corresponds to a scanning angle range of 0° →40° (rotating clockwise from 0° scanning position to 40° scanning position), and D11 corresponds to a scanning time range of 0-1.2 s. D12 corresponds to a scanning angle range of 40°→80° (rotating clockwise from 40° scanning position to 80° scanning position), and D12 corresponds to a scanning time range of 1.2-2.4 s. D13 corresponds to a scanning angle range of 80°→120° (rotating clockwise from 80° scanning position to 120° scanning position), and D13 corresponds to a scanning time range of 2.4-3.6 s. D14 corresponds to a scanning angle range of 120°→160° (rotating clockwise from 120° scanning position to 160° scanning position), and D14 corresponds to a scanning time range of 3.6-4.8 s. D15 corresponds to a scanning angle range of 160°→200° (rotating clockwise from 160° scanning position to 200° scanning position), and D15 corresponds to a scanning time range of 4.8-6 s. D21 corresponds to a scanning angle range of 200°→160° (rotating counterclockwise from 200° scanning position to 160° scanning position), and D21 corresponds to a scanning time range of 6-7.2 s. D22 corresponds to a scanning angle range of 160°→120° (rotating counterclockwise from 160° scanning position to 120° scanning position), and D22 corresponds to a scanning time range of 7.2-8.4 s. D23 corresponds to a scanning angle range of 120°→80° (rotating counterclockwise from 120° scanning position to 80° scanning position), and D23 corresponds to a scanning time range of 8.4-9.6 s. D24 corresponds to a scanning angle range of 80°→40° (rotating counterclockwise from 80° scanning position to 40° scanning position), and D24 corresponds to a scanning time range of 9.6-10.8 s. D25 corresponds to a scanning angle range of 40°→0° (rotating counterclockwise from 40° scanning position to 0° scanning position), and D25 corresponds to a scanning time range of 10.8-12 s. It can be seen that D11 and D25 correspond to the same scanning angle range but different scanning time ranges, D12 and D24 correspond to the same scanning angle range but different scanning time ranges, D13 and D23 correspond to the same scanning angle range but different scanning time ranges, D14 and D22 correspond to the same scanning angle range but different scanning time ranges, and D15 and D21 correspond to the same scanning angle range but different scanning time ranges. As shown in FIG. 10D, from the second reconstructed images I11-I25, it can be seen that D11 and D25, D12 and D24, D13 and D23, D14 and D22, D15 and D21 correspond to the same scanning region, respectively. It can be seen that S1 is partitioned in to D11-D15 corresponding to 5 different and contiguous scanning angle ranges, and 5 different and contiguous scanning time ranges, and S2 is partitioned in to D21-D25 corresponding to 5 different and contiguous scanning angle ranges, and 5 different and contiguous scanning time ranges. I11-I15 generated based on D11-D15 correspond to 5 different scanning regions, and I21-I25 generated based on D21-D25 correspond to 5 different scanning regions. The 5 scanning angle ranges corresponding to S1 are the same as the 5 scanning angle ranges corresponding to S2, and the 5 scanning regions corresponding to S1 are the same as the 5 scanning regions corresponding to S2. The spans of the scanning angle ranges of D11-D25 are the same, and the spans of the scanning time ranges of D11-D25 are the same.

In some embodiments, for each set of initial scanning data, the second reconstructed images corresponding to the plurality of sets of partitioned scanning data may be referred to as initial reconstruction data for the set of initial scanning data. As shown in FIG. 10D, the initial reconstruction data corresponding to the set of initial scanning data S1 includes the second reconstructed images I11-I15. The initial reconstruction data corresponding to the set of initial scanning data S2 includes the second reconstructed images I21-I25.

In some embodiments, the processing device 140 may not partition the initial scanning data, but directly reconstruct each set of initial scanning data to obtain initial reconstruction data. Referring to FIG. 10A and FIG. 10D, if the two sets of initial scanning data S1 and S2 are not partitioned and are directly reconstructed, the initial reconstruction data (e.g., an initial reconstructed image) R1 corresponding to S1 and the initial reconstruction data (e.g., an initial reconstructed image) R2 corresponding to S2 are generated, respectively, as shown in FIG. 10E.

In 930, the processing device 140 generates a third reconstructed image corresponding to a target time point by performing, in a chronological order, interpolating based on the initial reconstruction data of the plurality of sets of initial scanning data.

The target time point may be set by the user on his/her own according to the actual application and pre-stored in the medical imaging system 900-1 or the processing device 140. The target time point includes at least one time point.

After obtaining the plurality of sets of initial reconstruction data, the medical imaging system 900-1 or the processing device 140 generates the third reconstructed image corresponding to the target time point by performing, in the chronological order, temporal interpolating.

In some embodiments, the interpolation includes, but is not limited to, linear interpolation and nonlinear interpolation.

In some embodiments, for the case without partitioning, the processing device 140 may determine an initial time point corresponding to each set of initial reconstruction data. The initial time point corresponding to each set of initial reconstruction data may be a midpoint of the scanning time range of the corresponding set of initial scanning data. The processing device 140 may generate the third reconstructed image corresponding to the target time point by interpolating based on each set of initial reconstruction data and the corresponding initial time point.

Referring to FIGS. 10A, 10D, and 10E, the imaging device 110 acquires two sets of initial scanning data by performing two circles of short scanning through alternating rotation. A scanning angle range of each circle of short scanning is 0°-200°, and a span of the scanning time range of each circle of short scanning is 6 s. In a first circle, the imaging device rotates clockwise from 0° position to 200° position, with a scanning time range of 0-6 s, to obtain the first set of initial scanning data S1. In a second circle, the imaging device rotates counterclockwise from the 200° position to the 0° position, with a scanning time range of 6-12 s, to obtain the second set of initial scanning data S2. For the case without partitioning, the processing device 140 may generate a first set of initial reconstruction data R1 and a second set of initial reconstruction data R2 by reconstructing S1 and S2, respectively. The processing device 140 may determine initial time points corresponding to S1 and S2. For example, the initial time point corresponding to S1 is 3 s, and the initial time point corresponding to S2 is 9 s. The processing device 140 may generate the third reconstructed image corresponding to the target time point by interpolating based on R1 and R2 and their corresponding initial time points.

The embodiments of the present disclosure do not limit the specific manner of temporally interpolating based on different sets of initial reconstruction data, as long as the function can be realized. For example, for each pixel position, the processing device 140 may determine a relationship between time and the pixel value of the pixel position by fitting (e.g., linear fitting or non-linear fitting) the pixel values of the pixel position in the sets of initial reconstruction data and the initial time points of the sets of initial reconstruction data. The processing device 140 may input the target time point into the relationship to obtain the third reconstructed image corresponding to the target time point.

In some embodiments, in the case with partitioning, for each of the plurality of scanning regions and/or scanning angle ranges, the medical imaging system 900-1 or the processing device 140 may generate an intermediate reconstructed image for the scanning region and/or the scanning angle range corresponding to the target time point by performing, in the chronological order, interpolating based on the second reconstructed images corresponding to the scanning region and/or the scanning angle range. The medical imaging system 900-1 or the processing device 140 may generate the third reconstructed image corresponding to the target time point based on the intermediate reconstructed images for the plurality of projection regions and/or the plurality of scanning angle ranges corresponding to the target time point.

In some embodiments, as described above, the second reconstructed images and the partitioned scanning data corresponding to the same scanning angle range and/or same scanning region correspond to different scanning time ranges. For the second reconstructed images corresponding to the same scanning angle range and/or same scanning region, the processing device 140 may determine an initial time point corresponding to each second reconstructed image. The initial time point corresponding to each second reconstructed image may be a midpoint of a scanning time range of the corresponding set of partitioned scanning data. The processing device 140 may generate the intermediate reconstructed images of the same scanning angle range and/or same scanning region corresponding to the target time point by interpolating based on the second reconstructed images and the corresponding initial time points.

In some embodiments, after obtaining the intermediate reconstructed images for the scanning regions and/or scanning angle ranges corresponding to the target time point, the medical imaging system 900-1 or the processing device 140 may generate the third reconstructed image of the target subject for all the scanning regions and/or scanning angle ranges corresponding to the target time point by superimposing the intermediate reconstructed images. This embodiment does not limit the specific manner of superimposing the plurality of intermediate reconstructed images, as long as the function can be realized. For example, the processing device 140 may generate the third reconstructed image of the target time point by summing pixel values of corresponding pixels of the intermediate reconstructed images for the scanning regions and/or scanning angle ranges corresponding to the target time point.

The embodiments of the present disclosure do not limit the specific manner of temporally interpolating based on the second reconstructed images corresponding to the plurality of sets of partitioned scanning data to generate the intermediate reconstructed image of the target time point, as long as the function can be realized. Taking the second reconstructed images corresponding to a scanning angle range as an example, for each pixel position, the processing device 140 may determine a relationship between time and the pixel value of the pixel position by fitting (e.g., linear fitting or non-linear fitting) the pixel values of the pixel position in the second reconstructed images and the initial time points of the second reconstructed images. The processing device 140 may input the target time point into the relationship to obtain the intermediate reconstructed image corresponding to the scanning angle range and the target time point.

As shown in FIG. 10D, the processing device 140 may determine an initial time point corresponding to each of the second reconstructed images I11-I25. For example, the initial time point for I11 is 0.6 s, the initial time point for I12 is 1.8 s, the initial time point for I13 is 3 s, the initial time point for I14 is 4.2 s, the initial time point for I15 is 5.4 s, the initial time point for I21 is 6.6 s, the initial time point for I22 is 7.8 s, the initial time point for I23 is 9 s, the initial time point for I24 is 10.2 s, and the initial time point for I25 is 11.4 s. The processing device 140 may generate an intermediate reconstructed image M1 of a scanning region/scanning angle range of 0°-40° at the target time point by interpolating based on I11 and I25 and their corresponding initial time points. The processing device 140 may generate an intermediate reconstructed image M2 of a scanning region/scanning angle range of 40°-80° at the target time point by interpolating based on I12 and I24 and their corresponding initial time points. The processing device 140 may generate an intermediate reconstructed image M3 of a scanning region/scanning angle range of 80°-120° at the target time point by interpolating based on I13 and I23 and their corresponding initial time points. The processing device 140 may generate an intermediate reconstructed image M4 of a scanning region/scanning angle range of 120°-160° at the target time point by interpolating based on I14 and I22 and their corresponding initial time points. The processing device 140 may generate an intermediate reconstructed image M5 of a scanning region/scanning angle range of 160°-200° at the target time point by interpolating based on I15 and I21 and their corresponding initial time points. The processing device 140 may generate the third reconstructed image corresponding to the target time point by superimposing M1-M5.

For the sake of illustration, FIG. 10A, FIG. 10D, and FIG. 10E are illustrated with two circles of short scanning of alternating rotation. It can be understood that the above descriptions of FIG. 10A, FIG. 10D, and FIG. 10E are also applicable to full scanning, same direction rotation, and short scanning with two or more circles of alternating rotation.

In some embodiments of the present disclosure, by acquiring a plurality of sets of initial scanning data by performing a plurality of circles of full-angle scanning on the target subject, generating a plurality of sets of initial reconstruction data by reconstructing the plurality of sets of initial scanning data, and generating a third reconstructed image for a target time point by temporally interpolating the plurality of sets of initial reconstruction data, a plurality of sets of new reconstruction data (new reconstructed image) for the target time point can be generated, thereby increasing the count of samples for image reconstruction as well as the temporal resolution of dynamic imaging. Furthermore, in the case with partitioning, by superimposing the intermediate reconstructed images of the target subject for all scanning regions and/or scanning angle ranges corresponding to the target time point, the generated third reconstructed image of the target time point can include more information about the target subject, resulting in higher-quality reconstructed images.

In 940, the processing device 140 generates a first reconstructed image (a limited-angle reconstructed image) corresponding to the target time point based on the third reconstructed image and a portion of the plurality of sets of initial scanning data. The first reconstructed image may be a 2D image or a 3D image. The processing device 140 may set a plurality of target time points and generate a plurality of first reconstructed images, thereby enabling dynamic imaging of the target subject.

In some embodiments, the first reconstructed image may be reconstructed based on the third reconstructed image and a portion of the plurality of sets of initial scanning data using an iterative reconstruction algorithm.

The third reconstructed image corresponding to the target time point obtained by interpolation may deviate from the initial scanning data actually acquired at the target time point. The iterative reconstruction algorithm may be understood as using the actually acquired initial scanning data to iteratively update the third reconstructed image corresponding to the target time point, thereby outputting the first reconstructed image closer to the actually acquired initial scanning data. The portion of the plurality of sets of initial scanning data used for iteration reconstruction is referred to as correction scanning data.

In some embodiments, the correction scanning data may refer to the initial scanning data corresponding to a time period including the target time point.

In some embodiments, for the case with partitioning, the correction scanning data may refer to a set of partitioned scanning data of which the scanning time range includes the target time point. As shown in FIG. 10D, if the target time point is 8 s, D22 is identified as the correction scanning data.

In some embodiments, the correction scanning data may refer to initial scanning data corresponding to a time period including the target time point, and a span of a scanning time range corresponding to the correction scanning data is smaller than a span of a scanning time range corresponding to one set of partitioned scanning data. Further, the closer scanning time of the initial scanning data is to the target time point, the more accurately the initial scanning data can reflect the true attenuation value at the target time point, and thus, the correction scanning data may refer to initial scanning data at the target time point and initial scanning data within a time period adjacent to the target time point.

The time period adjacent to the target time point may include a certain time period immediately before the target time point and/or a certain time period immediately after the target time point. For example, correction scanning data is the initial scanning data corresponding to 5° immediately before and 5° immediately after the target time point. For example, as shown in FIG. 10D, if the target time point is 2.4 s, and the corresponding scanning angle is 80°, the correction scanning data may refer to the initial scanning data acquired in a range of 75°-85° around 2.4 s.

In some embodiments, the medical imaging system 900-1 or the processing device 140 may input the third reconstructed image of the target time point and the correction scanning data into a trained image reconstruction model, and generate the first reconstructed image of the target subject using the trained image reconstruction model.

The image reconstruction model may be a machine learning model. In some embodiments, the image reconstruction model may include a recurrent neural network (RNN) model, a deep neural network (DNN) model, a convolutional neural network (CNN) model, or the like, or any combination thereof.

In some embodiments, the medical imaging system 900-1 or the processing device 140 may input the third reconstructed image of the target time point, along with the correction scanning data, into the image reconstruction model, and the image reconstruction model outputs the first reconstructed image.

In some embodiments, the image reconstruction model may be trained using a plurality of third training samples with third labels. In some embodiments, the third training samples may include a plurality of sets of sample third reconstructed images and a plurality of sets of sample correction scanning data. The third labels corresponding to the third training samples may include sample first reconstructed images corrected for the sample third reconstructed images. The third training samples may be determined based on historical scanning data.

In some embodiments, the medical imaging system 900-1 or the processing device 140 may perform a training process similar to that used to train the first model to acquire the image reconstruction model. For more details, please refer to the relevant description described in the present disclosure.

In some embodiments of the present disclosure, by using an image reconstruction model to determine the first reconstructed image from the third reconstructed image, the self-learning capability of the machine learning model can be utilized to find patterns from a large amount of historical data, the correlation relationship among the third reconstructed image, the correction scanning data, and the first reconstructed image can be obtained, thus improving the accuracy and efficiency of determining the first reconstructed image, thereby ensuring the reconstruction speed.

As shown in FIG. 10F(a), the imaging device performs a plurality of circles of short scanning, a scanning angle range of each circle of short scanning is 0°-200°, and FIG. 10F(a) illustrates a set of initial scanning data (i.e., the gray area in FIG. 10F(a)) acquired by one circle of short scanning. The set of initial scanning data is partitioned into 5 sets of partitioned scanning data Pₐ₁-Pₐ₅ each of which is with an angle span of 40°. If the target time point is set to Tₐ, the correction scanning data corresponding to Tₐ may be the partitioned scanning data Pₐ₂ (i.e., the shaded area in FIG. 10F(a)) with an angle span of 40°.

As shown in FIG. 10F(b), the imaging device performs a plurality of circles of full scanning, a scanning angle range of each circle of full scanning is 0°-360°, and FIG. 10F(b) illustrates a set of initial scanning data (i.e., the gray area in FIG. 10F(b)) acquired by one circle of full scanning. The set of initial scanning data is partitioned into 5 sets of partitioned scanning data P_{b1}-P_{b5} each of which is with an angle span of 72°. If the target time point is set to T_{b}, the correction scanning data corresponding to T_{b} may be the partitioned scanning data P_{b1} (i.e., the shaded area in FIG. 10F(b)) with an angle span of 72°.

As shown in FIG. 10F(c), the imaging device performs a plurality of circles of short scanning, a scanning angle range of each circle of short scanning is 0°-200°, and FIG. 10F(c) illustrates a set of initial scanning data (i.e., the gray area in FIG. 10F(c)) acquired by one circle of short scanning. The set of initial scanning data is partitioned into 5 sets of partitioned scanning data Pₐ₁-Pₐ₅ each of which is with an angle span of 40°. If the target time point is set to Tₐ, the correction scanning data corresponding to Tₐ may be the initial scanning data corresponding to 5° immediately before and 5° immediately after Tₐ (i.e., the shaded area in FIG. 10F(c)), with an angle span of 10°.

As shown in FIG. 10F(d), the imaging device performs a plurality of circles of full scanning, a scanning angle range of each circle of full scanning is 0°-360°, and FIG. 10F(d) illustrates a set of initial scanning data (i.e., the gray area in FIG. 10F(d)) acquired by one circle of full scanning. The set of initial scanning data is partitioned into 5 sets of partitioned scanning data P_{b1}-P_{b5} each of which is with an angle span of 72°. If the target time point is set to T_{b}, the correction scanning data corresponding to T_{b} may be the initial scanning data corresponding to 5° immediately before and 5° immediately after T_{b} (i.e., the shaded area in FIG. 10F(d)), with an angle span of 10°.

As shown in FIG. 10F(e), the imaging device performs a plurality of circles of short scanning, a scanning angle range of each circle of short scanning is 0°-200°, and FIG. 10F(e) illustrates a set of initial scanning data (i.e., the gray area in FIG. 10F(e)) acquired by one circle of short scanning. The set of initial scanning data is not partitioned. If the target time point is set to Tₐ, the correction scanning data corresponding to Tₐ may be the initial scanning data corresponding to 5° immediately before and 5° immediately after Tₐ (i.e., the shaded area in FIG. 10F(e)), with an angle span of 10°.

As shown in FIG. 10F(f), the imaging device performs a plurality of circles of full scanning, a scanning angle range of each circle of full scanning is 0°-360°, and FIG. 10F(f) illustrates a set of initial scanning data (i.e., the gray area in FIG. 10F(f)) acquired by one circle of full scanning. The set of initial scanning data is not partitioned. If the target time point is set to T_{b}, the correction scanning data corresponding to T_{b} may be the initial scanning data corresponding to 5° immediately before and 5° immediately after T_{b} (i.e., the shaded area in FIG. 10F(f)), with an angle span of 10°.

As can be seen from FIG. 10F(a)-FIG. 10F(f), using the initial scanning data in the time period adjacent to the target time point as the correction scanning data can be applied to the case with partitioning and the case without partitioning. In addition, by using the initial scanning data in the time period adjacent to the target time point as the correction scanning data, the amount of the correction scanning data is relatively small, which allows for higher iterative reconstruction speed, lower computational load, higher temporal resolution, and more temporal sampling points while ensuring reconstruction quality.

In some embodiments, the selection of the target time point needs to meet the following conditions. (1) Actual acquisition has been performed at the target time point (i.e., initial scanning data exists at the target time point). For example, if the scanning time range of the plurality of sets of initial scanning data is 0-12 s, a time point other than 0-12 s (e.g., 13 s) cannot be used as the target time point. (2) Real projection data (initial scanning data) exists at a certain angle range around the target time point. For example, a time point t corresponds to 2° in the first circle of full-angle scanning, and if initial scanning data corresponding to 5° immediately before and 5° immediately after the target time point is needed to be designated as the correction scanning data, the time point t cannot be used as the target time point. (3) The correction scanning data corresponding to each target time point needs to be non-overlapping, otherwise, reconstruction results may be inaccurate if the same initial scanning data is utilized to update third reconstructed images corresponding to different target time points. For example, if the correction scanning data refers to a set of partitioned scanning data of which the scanning time range includes the target time point, the maximum count of target time points is equal to the count of all sets of partitioned scanning data, and each set of partitioned scanning data only corresponds to one target time point. As another example, if the correction scanning data refers to the initial scanning data at the target time point and the initial scanning data corresponding to 5° immediately before and 5° immediately after the target time point, in the case of the FIG. 1D, a difference of scanning angles corresponding to two adjacent target time points needs to be greater than or equal to 10°, or a time difference between the two adjacent target time points needs to be greater than or equal to 0.3 s, so as to ensure that the correction scanning data corresponding to each target time point does not overlap.

FIG. 10D is taken as an example to illustrate Example 1, Example 2, and Example 3.

In Example 1, the imaging device obtains a set of initial scanning data by performing a circle of short scanning with 0-200° every 6 s, and generates a set of initial reconstruction data by reconstructing the set of initial scanning data, which can realize a temporal resolution of 6 s/frame.

In Example 2, if the correction scanning data refers initial scanning data at the target time point and initial scanning data corresponding to 5° immediately before and 5° immediately after the target time point, a difference of scanning angles corresponding to two adjacent target time points needs to be greater than or equal to 10°, or a time difference between the two adjacent target time points needs to be greater than or equal to 0.3 s, thus the maximum temporal resolution can be achieved at 0.3 s/frame.

In Example 3, if the correction scanning data refers to a set of partitioned scanning data of which the scanning time range includes the target time point, at most one target time point can be selected in each set of partitioned scanning data, which can realize a maximum temporal resolution of 1.2 s/frame. If the count of the sets of partitioned scanning data is increased, for example, if each set of initial scanning data is partitioned in accordance with 10°, the highest temporal resolution that can be realized can theoretically be consistent with Example 2. However, if the corresponding scanning angle range of each set of partitioned scanning data is too small, the second reconstructed image generated after reconstruction is not of high image quality, which may lead to the subsequent intermediate reconstructed image obtained by interpolation, and even the third reconstructed image at the target time point not being of high image quality, thereby affecting the iterative reconstruction results. At the same time, too many sets of partitioned scanning data increase the computation load.

Clearly, defining correction scanning data as the initial scanning data at the target time point and the initial scanning data within a time period adjacent to the target time point greatly improves the temporal resolution. Additionally, the selection of the adjacent time period determines the iterative reconstruction effect as well as the temporal resolution. The smaller the selected adjacent time period, the higher the temporal resolution that can be achieved. However, if the amount of correction scanning data is too small, it may affect the iterative reconstruction effect. Therefore, the scanning angle range corresponding to the time period adjacent to the target time point of the correction scanning data may be minimized while ensuring sufficient data volume to satisfy the iterative reconstruction effect. Preferably, the initial scanning data within 5° immediately before and 5° immediately after the target time point may be selected as the correction scanning data.

In some embodiments, the medical imaging system 900-1 or the processing device 140 may determine the scanning angle range and/or the scanning time range corresponding to the initial scanning data (i.e., the corrected scanning data) within a time period adjacent to the target time point using a second model. The second model may be a machine learning model. In some embodiments, the second model may include a recurrent neural network (RNN) model, a deep neural network (DNN) model, a convolutional neural network (CNN) model, or the like, or any combination thereof.

In some embodiments, the medical imaging system 900-1 or the processing device 140 may input a temporal resolution requirement, a quality requirement of the target reconstructed image, a quality requirement of the first reconstructed image, a set of partitioned projection data of which the scanning time range includes the target time point, the target time point, and the third reconstructed image corresponding to the target time point into the second model, and the second model outputs the scanning angle range and/or the scanning time range of the correction scanning data.

In some embodiments, the input of the second model may also include imaging information, an imaging portion, and contrast agent information. For more descriptions of the imaging information and contrast agent information, please refer to the relevant description in the first model.

In some embodiments, the second model may be trained by a plurality of second training samples with second label data. In some embodiments, the second training samples may include sample temporal resolution requirements, quality requirements of sample target reconstructed images, quality requirements of sample first reconstructed images, sample partitioned projection data corresponding to sample target time points, sample target time points, and sample third reconstructed images corresponding to the sample target time points. In other embodiments, the second training samples may include sample temporal resolution requirements, quality requirements of sample target reconstructed images, quality requirements of sample first reconstructed images, sample partitioned projection data corresponding to sample target time points, sample target time points, sample third reconstructed images corresponding to the sample target time points, sample imaging information, sample imaging portions, sample contrast agent information, or the like. The second label data corresponding to the second training sample may include a scanning angle range and/or a scanning time range corresponding to the correction scanning data for each sample target time point. The second training samples may be determined based on historical scanning data.

In some embodiments, for each of the second training samples, the medical imaging system 900-1 or the processing device 140 may acquire a plurality of candidate scanning angle/time ranges corresponding to the initial scanning data within a time period adjacent to the sample target time point, calculate a second feature corresponding to each candidate scanning angle/time range, determine a second score for each candidate scanning angle/time range based on the second feature, and select a candidate scanning angle/time range with the highest second score as the second label data corresponding to the second training sample. The second feature may include at least one of an image quality of the sample first reconstructed image, an image quality of the sample target reconstructed image, a reconstruction speed, and a computation load obtained based on the candidate scanning angle/time range. The higher the image quality of the sample first reconstructed image, the higher the image quality of the sample target reconstructed image, the faster the reconstruction speed, and the smaller the computational load, the higher the second score.

The training process for training the second model is similar to the training process for training the first model, which is not repeated herein.

In some embodiments of the present disclosure, by using a trained second model to determine the scanning angle/time range corresponding to the initial scanning data (i.e., the correction scanning data) within the time period adjacent to the target time point, the self-learning capability of the machine learning model can be utilized to find patterns from a large amount of historical data, which improves the efficiency and reasonableness of determining the scanning angle/time range corresponding to the initial scanning data (i.e., the correction scanning data) within the time period to adjacent the target time point, thereby ensuring both the reconstruction quality and temporal resolution, as well as the reconstruction speed.

In some embodiments, each target time point corresponds to different real projection data, and thus, for different target time points, the medical imaging system 900-1 or the processing device 140 may determine a scanning angle/time range corresponding to initial scanning data (i.e., correction scanning data) in a time period adjacent to each target time point, further improving the reasonableness and accuracy of the scanning angle/time range determination.

In some embodiments of the present disclosure, the first reconstructed image is generated by iteratively reconstructing the third reconstructed image at the target time point based on the initial scanning data (i.e., the correction scanning data) in a time period adjacent to the target time point. In this way, performing iteration on the third reconstructed image at the target time point based on the initial scanning data (i.e., the correction scanning data) in the time period adjacent to the target time point, not only improves the reconstruction speed and the temporal resolution, but also ensures the image quality of the reconstructed image.

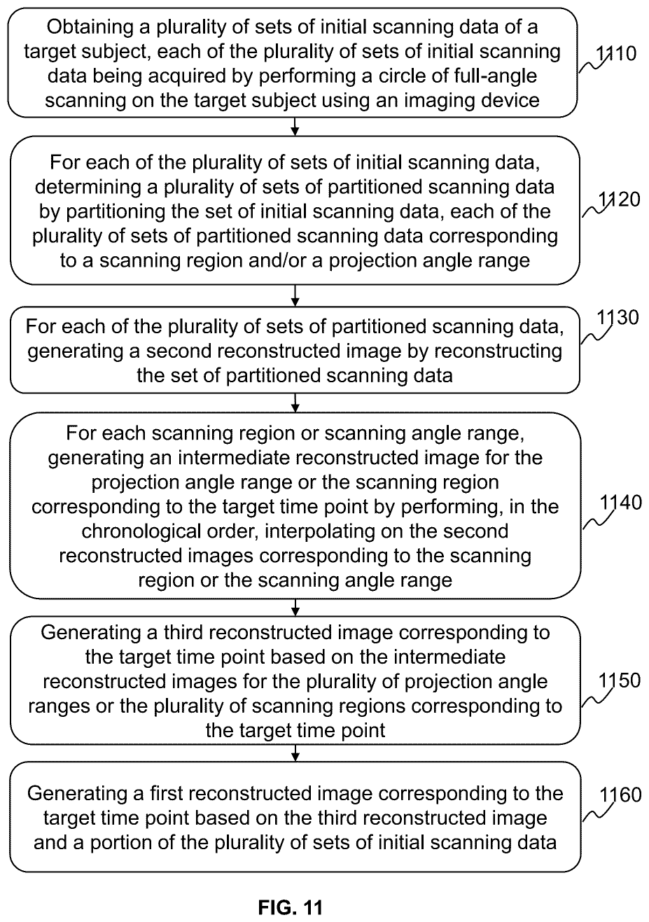

FIG. 11 is a flowchart illustrating an exemplary process for obtaining a first reconstructed image according to other embodiments of the present disclosure. As shown in FIG. 11, process 1100 includes the following operations. In some embodiments, process 1100 may be performed by the medical imaging system 900-1 or the processing device 140.

In 1110, a plurality of sets of initial scanning data is obtained. Each of the plurality of sets of initial scanning data is acquired by performing a circle of full-angle scanning on a target subject using an imaging device.

In 1120, for each of the plurality of sets of initial scanning data, a plurality of sets of partitioned scanning data is determined by partitioning the set of initial scanning data. Each of the plurality of sets of partitioned scanning data corresponds to a scanning region and/or a projection angle range.

In 1130, for each of the plurality of sets of partitioned scanning data, a second reconstructed image is generated by reconstructing the set of partitioned scanning data.

In 1140, for the second reconstructed images corresponding to the same scanning region or the same scanning angle range, an intermediate reconstructed image for the projection angle range or the scanning region corresponding to a target time point is generated by performing, in the chronological order, interpolating.

In 1150, a third reconstructed image corresponding to the target time point is generated based on the intermediate reconstructed images for the plurality of projection angle ranges or the plurality of scanning regions corresponding to the target time point.

In 1160, a first reconstructed image is reconstructed based on the third reconstructed image and a portion of the plurality of sets of initial scanning data.

More descriptions for operations 1110-1160 can be found in FIGS. 9B-10F and the descriptions thereof.

In some embodiments, when obtaining the first reconstructed image in operations 940 or 1160, since the iterative reconstruction uses real projection data (i.e., the correction scanning data) within limited-angles, significant limited-angle artifacts are produced in the first reconstructed image (limited-angle reconstructed image). The processing device 140 may generate an artifact-optimized target reconstructed image by performing an artifact optimization process on the first reconstructed image. For example, the processing device 140 may generate the artifact-optimized target reconstructed image by performing a limited-angle artifact optimization process on the first reconstructed image using singular value decomposition. Alternatively, the processing device 140 may generate the artifact-optimized target reconstructed image using the method described in FIG. 4-FIG. 8B to input the limited-angle reconstructed image into the trained deep learning model 500 to perform optimization processing of the limited-angle artifacts in the limited-angle reconstructed image. The embodiments of the present disclosure does not limit the specific artifact optimization method, as long as the functions can be realized.

FIG. 12A is a block diagram illustrating an exemplary medical imaging system according to other embodiments of the present disclosure. In some embodiments, as shown in FIG. 12A, a medical imaging system 1200-1 includes a projection acquisition module 1201, a combination module 1202, and a generation module 1203. The medical imaging system 1200-1 may be implemented on the processing device 140 through hardware or software.

The projection acquisition module 1201 is configured to acquire at least two sets of projection data of a target subject via an imaging device (e.g., the imaging device 110).

The combination module 1202 is configured to determine combined projection data based on the at least two sets of projection data.

The generation module 1203 is configured to generate a first reconstructed image based on the combined projection data.

More descriptions can be found in FIGS. 12B-14B and their related descriptions.

It should be noted that the above description of the medical imaging system 1200-1 and its modules is for convenience of description only and should not limit the present disclosure to the scope of the illustrated embodiments. It is understood that for those skilled in the art, after understanding the principles of the system, they may combine the modules arbitrarily or form subsystems connected to other modules without departing from these principles. In some embodiments, the projection acquisition module 1201, the combination module 1202, and the generation module 1203 described in FIG. 12A may be different modules within one system, or one module may implement the functions of two or more of the above modules. For example, the modules may share a storage module, or the modules may each have their own respective storage modules. Such variations are all within the scope of the present disclosure.

FIG. 12B is a flowchart illustrating an exemplary process for obtaining a first reconstructed image according to other embodiments of the present disclosure. As shown in FIG. 12B, process 1200-2 includes the following operations. In some embodiments, process 1200-2 may be performed by the medical imaging system 1200-1 or the processing device 140.

In 1210, the processing device 140 acquires at least two sets of projection data of a target subject using an imaging device.

In some embodiments, the imaging device (e.g., the imaging device 110) includes at least two radiation sources and at least one detector. Each set of projection data corresponds to one radiation source. The at least two sets of projection data correspond to different projection angle ranges. The at least two sets of projection data are acquired within a same time period.

The projection data (also referred to as initial scanning data) refers to data recorded by the detector receiving rays emitted by the radiation source(s) during imaging. The projection data may be transmitted in real-time to the medical imaging system 1200-1 or the processing device 140 after scanning by the imaging device. Alternatively, after being obtained by the imaging device, the projection data may be stored in a corresponding storage component (e.g., the storage device 250). The medical imaging system 1200-1 or the processing device 140 directly retrieves the projection data from the corresponding storage component of the imaging device when needed. The projection data may also be obtained by the medical imaging system 1200-1 or the processing device 140 from the imaging device's post-processing workstation or from servers such as picture archiving and communication systems (PACS). This embodiment does not limit the specific manner of acquiring the projection data, as long as the function can be achieved.

In some embodiments, the medical imaging system 1200-1 or the processing device 140 controls a plurality of radiation sources of the imaging device to scan a same region of interest (ROI) of the target subject simultaneously to acquire at least two sets of projection data of the target subject. One radiation source rotates a certain angle range and emits a beam to the target subject. The corresponding detector receives the beam and generates one set of projection data. The at least two sets of projection data correspond to different radiation sources.

In some embodiments, the plurality of radiation sources rotate a certain angle range and emit beams to the target subject within the same time period to acquire the at least two sets of projection data, hence, the at least two sets of projection data are acquired concurrently.

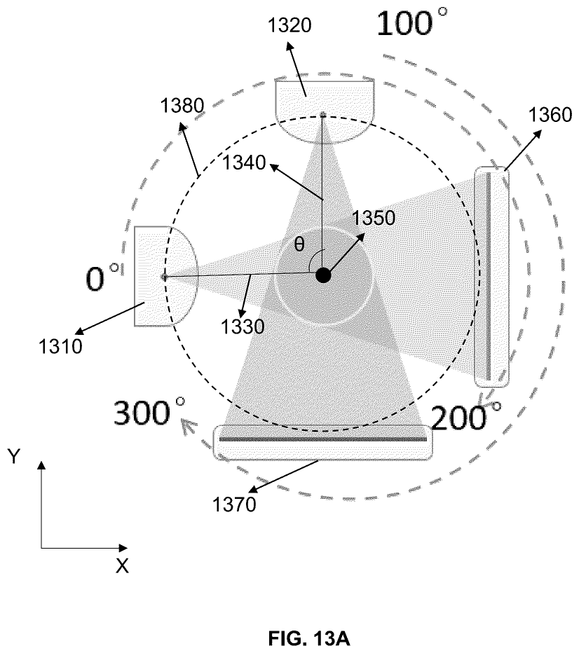

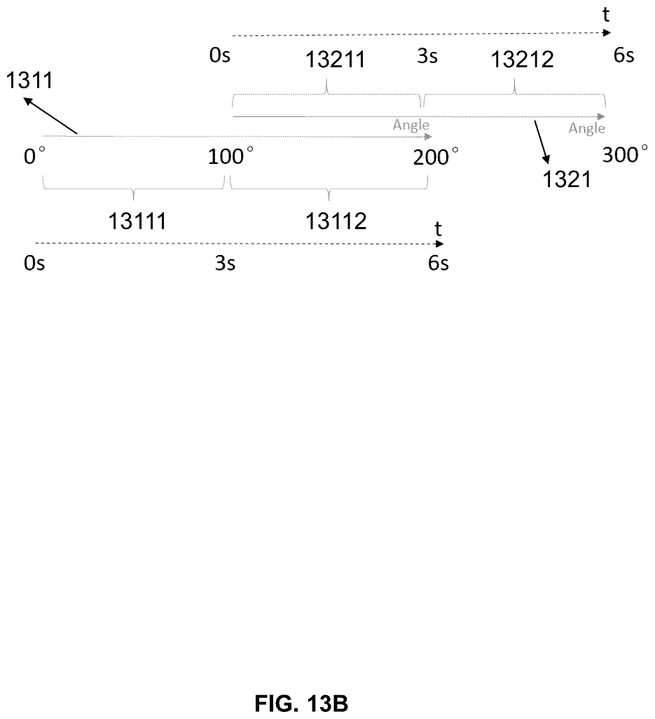

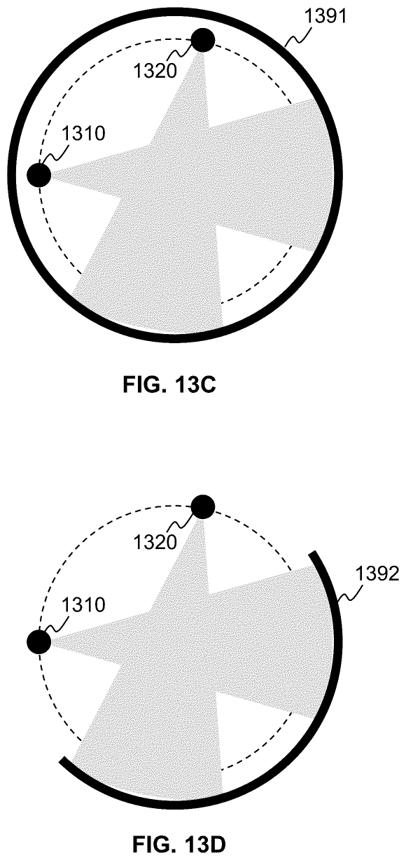

FIG. 13A is a schematic diagram illustrating an exemplary scanning process of an imaging device according to some embodiments of the present disclosure. As shown in FIG. 13A, the imaging device includes two radiation sources (e.g., a first radiation source 1310 and a second radiation source 1320) and two detectors (e.g., a first detector 1360 corresponding to the first radiation source 1310 and a second detector 1370 corresponding to the second radiation source 1320). It should be noted that the radiation sources shown in FIG. 13A are only exemplary, and that the imaging device 110 may also include more radiation sources, such as 3 radiation sources or 4 radiation sources, without limitation herein.

In some embodiments, the at least two radiation sources are spaced apart. Taking FIG. 13A as an example, the first radiation source 1310 and the second radiation source 1320 being spaced apart refers that a line 1330 connecting the first radiation source 1310 and a rotation center 1350 of a rotation path 1380 and a line 1340 connecting the second radiation source 1320 and the rotation center 1350 form a certain angle θ (e.g., as shown in FIG. 13A, θ is 100°).

In some embodiments, the imaging device 110 includes one detector, i.e., the plurality of radiation sources may correspond to a single detector. In some embodiments, when the imaging device 110 includes one detector, the detector may be in a form of a ring (e.g., a detector 1391 as shown in FIG. 13C), so as to facilitate complete reception of rays generated by the radiation sources. In some embodiments, when the imaging device 110 includes one detector, the detector may also have a large arc (e.g., the detector's arc is capable of completely covering the scanning angles of the at least two radiation sources, etc.) (e.g., a detector 1392 as shown in FIG. 13D).

In some embodiments, the imaging device 110 includes at least two detectors. The at least two radiation sources may have a one-to-one correspondence with the at least two detectors (e.g., the radiation sources 1310 and 1320, and detectors 1360 and 1370 as shown in FIG. 13A).

A plurality of radiation sources and at least one detector may be mounted on a C-arm or may be mounted on a ring gantry. For example, the imaging device 110 includes a first radiation source and a first detector corresponding to the first radiation source, and a second radiation source and a second detector corresponding to the second radiation source. The first radiation source and the first detector are mounted on a first C-arm, and the second radiation source and the second detector are mounted on a second C-arm. The first C-arm is fixed to the floor, and the second C-arm is fixed to the ceiling.

In some embodiments, the at least two sets of projection data correspond to different projection angle ranges (also referred to as scanning angle ranges).

The projection angle refers to a scanning angle position that the radiation source passes through in the process of acquiring a set of projection data. Multiple scanning angle positions are included in a rotation path of the radiation source. For example, as shown in FIG. 13A, if a direction from the rotation center 1350 to a scanning angle position is in an opposite direction of the X-axis as shown in FIG. 1 or FIG. 2, the scanning angle position corresponds to 0° position (e.g., 9 o'clock position). Along the clockwise direction, if a direction from the rotation center 1350 to a scanning angle position is the positive direction of Y-axis as shown in FIG. 1 or FIG. 2, the scanning angle position (e.g., 12 o'clock position) may be designated as 90° position, and so on. The projection data acquired by rotating the radiation source from 0° position to 100° position corresponds to a projection angle range of 0°-100°.

In some embodiments, spans (i.e., a difference between the maximum and minimum values in the projection angle range) of projection angle ranges of at least two sets of projection data may be the same. For example, as shown in FIG. 13B, a first set of projection data 13111 corresponding to the first radiation source and a second set of projection data 13211 corresponding to the second radiation source are acquired within 0-3 s, the first set of projection data 13111 has a projection angle of 0°-100°, and the second set of projection data 13211 has a projection angle of 100°-200°. Thus, each of the two sets of projection data has a span of projection angle range of 100°.

In some embodiments, the at least two radiation sources spaced apart may be rotated synchronously to acquire the at least two sets of projection data corresponding to different projection angle ranges in the same time period. Synchronized rotation of the at least two radiation sources refers to that the at least two radiation sources are rotated at the same time and always maintain the constant spacing distance between each other. For example, as shown in FIG. 13A, the first radiation source 1310 and the second radiation source 1320 are disposed with an angle difference of 100°, and during a time period T, the first radiation source 1310 and the second radiation source 1320 synchronously rotate by 100°. For example, during the time period T, the first radiation source 1310 rotates from a scanning angle position of 0° to a scanning angle position of 100° and emits first rays, and at the same time, the second radiation source 1320 rotates from a scanning angle position of 100° to a scanning angle position of 200° and emits second rays. The first detector 1360 receives the first rays to obtain a first set of projection data, and the first set of projection data corresponds to a projection angle range of 0°-100°. The second detector 1370 receives the second rays to obtain a second set of projection data, and the second set of projection data corresponds to a projection angle range of 100°-200°.

In some embodiments, the at least two radiation sources spaced apart may acquire the at least two sets of projection data corresponding to different projection angle ranges by nonsynchronous rotation. For example, during the same time period, rotational speeds and/or rotational directions of the at least two radiation sources may be different.

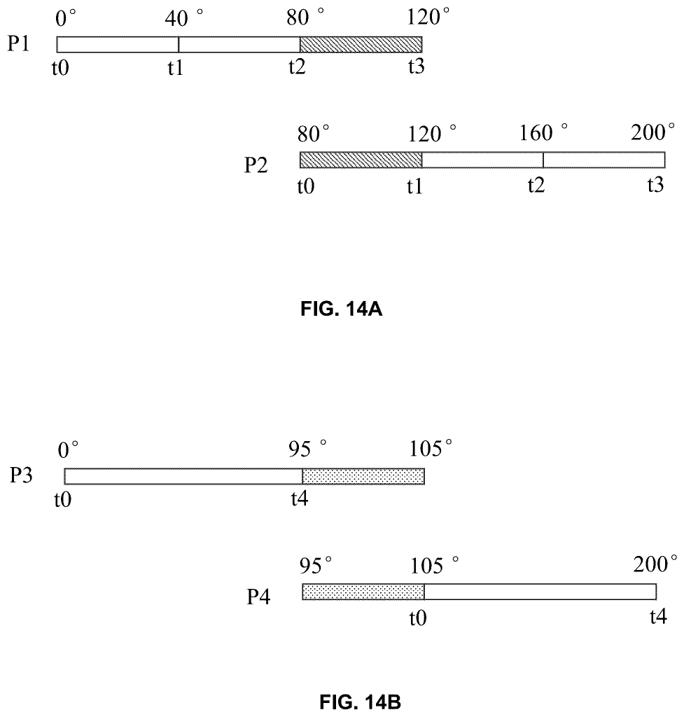

In some embodiments, the different projection angle ranges corresponding to the at least two sets of projection data partially overlap. For example, as shown in FIG. 14A, the two radiation sources are set 80° apart, and the first radiation source and the second radiation source rotate synchronously by 120°. For example, in t0-t3, the first radiation source rotates from a scanning angle position of 0° to a scanning angle position of 120° and emits the first rays, and at the same time, the second radiation source rotates from a scanning angle position of 80° to a scanning angle position of 200° and emits the second rays. The first detector receives the first rays to obtain a first set of projection data P1, and the first set of projection data corresponds to a first projection angle range of 0°-120°. The second detector receives the second rays to obtain a second set of projection data P2, and the second set of projection data corresponds to a second projection angle range of 80°-200°. There is an overlapping projection angle range of 80°-120° between the first projection angle range 0°-120° and the second projection angle range 80°-200°.

In some embodiments, the different projection angle ranges of the at least two sets of projection data are contiguous. The projection angle ranges being contiguous refers that the projection angle ranges of the at least two radiation sources are non-overlapping and gapless. For example, referring to FIG. 13A and FIG. 13B, the first radiation source 1310 and the second radiation source 1320 are set 100° apart, and the first radiation source 1310 and the second radiation source 1320 rotate synchronously by 100°. For example, in 0-3 s, the first radiation source 1310 rotates from a scanning angle position of 0° to a scanning angle position of 100° and emits first rays, and simultaneously, the second radiation source 1320 rotates from a scanning angle position of 100° to a scanning angle position of 200° and emits second rays. The first detector 1360 receives the first rays to obtain a first set of projection data, and the first set of projection data corresponds to a first projection angle range of 0°-100°. The second detector 1370 receives the second rays to obtain a second set of projection data, and the second set of projection data corresponds to a second projection angle range of 100°-200°. The first projection angle range 0°-100° and the second projection angle range 100°-200° are contiguous and there is no overlap nor gap between the first projection angle range and the second projection angle range.

In some embodiments, an angle interval exists between the different projection angle ranges of the at least two sets of projection data. The angle interval between the different projection angle ranges refers to an angle range cannot be scanned by the emitted rays. For example, as shown in FIG. 14B, the two radiation sources are set at 105° apart, and the first radiation source and the second radiation source rotate synchronously by 95°. For example, in t0-t4, the first radiation source rotates from a scanning angle position of 0° to a scanning angle position of 95° and emits the first rays, and at the same time, the second radiation source rotates from a scanning angle position of 105° to a scanning angle position of 200° and emits the second rays. The first detector receives the first rays to obtain a first set of projection data P3, and the first set of projection data corresponds to a first projection angle range 0°-95°. The second detector receives the second rays to obtain a second set of projection data P4, and the second set of projection data corresponds to a second projection angle range 105°-200°. There is an angle interval of 95°-105° between the first projection angle range 0°-95° and the second projection angle range 105°-200°.

In some embodiments, the medical imaging system 1200-1 or the processing device 140 may control at least two radiation sources of the imaging device to emit rays for scanning simultaneously. As shown in FIG. 13A, the medical imaging system 1200-1 or the processing device 140 may simultaneously control the first radiation source 1311 to start scanning at a scanning start angle of 0°, the second radiation source 1321 to start scanning at a scanning start angle of 100°, the first radiation source 1311 to end scanning at a scanning termination angle of 100°, and the second radiation source 1321 to end scanning at a scanning termination angle of 200°.

In some embodiments, the at least two radiation sources emit rays at the same energy level to acquire the at least two sets of projection data. For example, tube voltages of the at least two radiation sources may be the same (e.g., the energy levels of the at least two radiation sources are both 80 kV or 120 kV). In some embodiments of the present disclosure, emitting rays at the same energy level from the at least two radiation sources when acquiring projection data ensures that a deviation between the acquired different sets of projection data is smaller and the determined combined projection data is more accurate, thereby further improving the image quality of the subsequently generated images.

In 1220, the processing device 140 determines combined projection data based on the at least two sets of projection data.

The combined projection data refers to projection data obtained by splicing or combining the plurality of sets (e.g., at least two sets) of projection data.

In some embodiments, when the projection angle ranges of the at least two sets of projection data are contiguous, the medical imaging system 1200-1 or the processing device 140 may combine the projection data from the different radiation sources to determine the combined projection data. For example, as in FIG. 13B, the projection angle range of the first set of projection data corresponding to the first radiation source is 0°-100°, and the projection angle range of the second set of projection data corresponding to the second radiation source is 100°-200°. The medical imaging system 1200-1 or the processing device 140 may combine the projection data corresponding to the first radiation source and the projection data corresponding to the second radiation source to determine combined projection data with a projection angle range of 0°-200°. In some embodiments of the present disclosure, when the projection angle ranges are contiguous, acquiring the projection data by at least two radiation sources can increase the count of projection data samples and scanning speed.

In some embodiments, when the different projection angle ranges corresponding to the at least two sets of projection data partially overlap, the medical imaging system 1200-1 or the processing device 140 may obtain overlapping projection data by averaging the portions of the at least two sets of projection data where the projection angle ranges overlap. The medical imaging system 1200-1 or the processing device 140 may determine the portions of the at least two sets of projection data in which the projection angle ranges do not overlap and the overlapping projection data as the combined projection data.

For example, as shown in FIG. 14A, the first set of projection data D1 corresponds to a projection angle range of 0°-120°, the second set of projection data D2 corresponds to a projection angle range of 80°-200°, and the projection angle ranges of the first set of projection data and the second set of projection data have an overlapping projection angle range of 80°-120°. The medical imaging system 1200-1 or the processing device 140 may obtain the overlapping projection data by performing arithmetic averaging on the projection data corresponding to the overlapping projection angle ranges. For example, the medical imaging system 1200-1 or the processing device 140 may arithmetically average the projection data corresponding to 80° in the first set of projection data with the projection data corresponding to 80° in the second set of projection data to obtain overlapping projection data corresponding to 80°, and arithmetically average the projection data corresponding to 81° in the first set of projection data with the projection data corresponding to 81° in the second set of projection data to obtain overlapping projection data corresponding to 81°, and so on, to obtain overlapping projection data corresponding to 80°-120°.

In some embodiments, the medical imaging system 1200-1 or the processing device 140 may obtain the overlapping projection data by performing weighted averaging on the projection data corresponding to the overlapping projection angle ranges. Weights may be related to the magnitude of motion of the target subject, i.e., the greater the magnitude of motion of the target subject, the smaller the weight of the corresponding projection data. As mentioned above, the projection angle ranges of the angle overlapped projection data are the same, but their corresponding scanning time ranges are different, and the motion of the target subject (e.g., patient) may be different. For example, as shown in FIG. 14, the first set of projection data P1 is collected in t0-t3, corresponding to a projection angle range of 0°-120°, and the second set of projection data D2 is collected in t0-t3, corresponding to a projection angle range of 80°-200°. There is an overlapping projection angle range of 80°-120° between the projection angle range of the first set of projection data and the projection angle range of the second set of projection data. In the first set of projection data, the projection data corresponding to 80°-120° is collected within t2-t3, and in the second set of projection data, the projection data corresponding to 80°-120° is collected within t0-t1. The target subject keeps still during the time period t0-t1, but may move during the time period t2-t3. The greater the motion of the target subject, the greater the impact on the imaging quality may be, and the corresponding projection data may be less accurate. In some embodiments, the magnitude of motion of the target subject may be obtained in various ways. For example, the magnitude of motion of the target subject may be obtained in one or more ways, such as by an electrocardiogram, a respiratory curve, a surveillance video, etc.

For example, the weight corresponding to the first set of projection data is W1, the weight corresponding to the second set of projection data is W2, where W1, W2∈[0,1]. The medical imaging system 1200-1 or the processing device 140 may determine the overlapping projection data corresponding to 80° by weighted averaging the projection data corresponding to 80° in the first set of projection data and the projection data corresponding to 80° in the second set of projection data based on W1 and W2. The medical imaging system 1200-1 or the processing device 140 may determine the overlapping projection data corresponding to 81° by weighted averaging the projection data corresponding to 81° in the first set of projection data and the projection data corresponding to 81° in the second set of projection data based on W1 and W2, and so on, to determine the overlapping projection data corresponding to 80°-120°.

In some embodiments, weighted averaging the angle overlapped projection data based on the motion of the target subject can reduce the effect of the motion of the target subject on the projection data, and improve the accuracy of the generated image.

In some embodiments, the medical imaging system 1200-1 or the processing device 140 may determine the portion of the at least two sets of projection data in which the projection angle ranges do not overlap and the overlapping projection data as the combined projection data. For example, as shown in FIG. 14, the medical imaging system 1200-1 or the processing device 140 may determine the projection data corresponding to 0°-80° in the first set of projection data, the overlapping projection data corresponding to 80°-120°, and the projection data corresponding to 120°-200° in the second set of projection data as the combined projection data with a projection angle range of 0° to 200°.

In some embodiments of the present disclosure, by obtaining the overlapping projection data by averaging the portions of the at least two sets of projection data in which the projection angle ranges overlap, and further obtaining the combined projection data, it is possible to reduce the noise interference and improve the accuracy of the obtained projection data. Furthermore, more sampling data and a larger projection angle range can be obtained, improving the image quality of the subsequently generated images.

In some embodiments, when an angle interval exists between projection angle ranges of different radiation sources, the medical imaging system 1200-1 or the processing device 140 may determine, based on the at least two sets of projection data, estimated projection data corresponding to the angle interval, and combine the at least two sets of projection data and the estimated projection data as the combined projection data. In some embodiments, when the angle interval is large, the estimated projection data may be less accurate, resulting in the image quality of the generated image being compromised, and thus the angle interval is typically small. For example, the angle interval may be between 5° and 10°.

The estimated projection data refers to projection data corresponding to the angle interval predicted based on an algorithm or a model, rather than the real projection data generated by receiving rays by the detector.

In some embodiments, the medical imaging system 1200-1 or the processing device 140 may determine the estimated projection data by interpolation (e.g., linear interpolation, nonlinear interpolation, etc.) based on the at least two sets of projection data.

In some embodiments, the medical imaging system 1200-1 or the processing device 140 may also determine the estimated projection data based on the at least two sets of projection data using a machine learning model.

Exemplary machine learning models may include a neural network model, a deep neural network model, a recurrent neural network model, etc., or a combination thereof.

In some embodiments, an input of the machine learning model includes the projection data corresponding to the at least two sets of radiation sources and their corresponding projection angle ranges, and an output of the machine learning model includes the estimated projection data. In some embodiments, the machine learning model may be acquired by training. The training samples include a large amount of sample projection data with angle intervals present and sample projection angle ranges, and the labels include sample estimated projection data. For example, projection data of 0°-200° may be acquired, of which projection data of 0°-80° and 100°-200° are used as the training sample, and projection data of 80°-100° is used as the corresponding label.

As shown in FIG. 14B, the first set of projection data P3 corresponds to a projection angle range of 0°-95°, the second set of projection data P4 corresponds to a projection angle range of 105°-200°, and the projection angle range of the first set of projection data D3 and the projection angle range of the second set of projection projection data D4 have an angle interval of 95°-105°. The medical imaging system 1200-1 or the processing device 140 may determine the estimated projection data corresponding to 95°-105° based on the two sets of projection data, and then determine projection data corresponding to 0°-95° in the first set of projection data P3, projection data corresponding to 105°-200° in the second set of projection data P4, and the estimated projection data corresponding to 95°-105° as the combined projection data corresponding to 0°-200°.

In some embodiments of the present disclosure, the presence of the angle interval between the projection angle ranges of the different radiation sources reduces an angle range that needs to be scanned and improves the imaging speed.

By determining the combined projection data based on the at least two sets of projection data, a plurality of sets of projection data corresponding to smaller angle ranges can be combined into a set of projection data corresponding to a larger angle range, and more projection data and a larger projection angle range can be achieved, thereby improving the image quality and efficiency of subsequently reconstructed images.

Exemplarily, it is assumed that the generation of an image requires the acquisition of projection data of at least 200° (i.e., the corresponding projection angle range has a span of 200°).

When there is only one radiation source, the radiation source needs to rotate for 6 s to acquire projection data with a projection angle span of 200° to generate an image. In that case, it can be understood that the imaging device may acquire projection data for generating an image every 6 s.

The embodiment of determining the combined projection data shown in FIG. 13B corresponds to the embodiment of the scanning process of at least two radiation sources shown in FIG. 13A. As shown in FIG. 13B, the medical imaging system 1200-1 or the processing device 140 controls the synchronized rotation of the first radiation source 1310 and the second radiation source 1320 by 200° in 0-6 s. In 0 s˜3 s, the first radiation source 1310 acquires a first projection data 13111 with a projection angle range of 0° ˜100°, and the second radiation source 1320 acquires a third projection data 13211 with a projection angle range of 100° ˜200°. In 3 s˜6 s, the first radiation source 1310 acquires a second projection data 13112 with a projection angle range of 100° ˜200°, and the second radiation source 1320 acquires a forth projection data 13212 with a projection angle range of 200° ˜300°. The medical imaging system 1200-1 or the processing device 140 may combine the first projection data 13111 and the third projection data 13211 to generate first combined projection data of 0° ˜200° for generating a first image, and combine the second projection data 13112 and the fourth projection data 13212 to generate second combined projection data of 100° ˜300° for generating a second image. In this way, it can be understood that the imaging device may acquire projection data for generating an image every 3 s. In some embodiments of the present disclosure, by the method described above, the scanning time for acquiring projection data for generating one image is greatly reduced, and, for static imaging, the efficiency of the image generation is increased, and, for dynamic imaging, the temporal resolution of the dynamic image is improved.

The above description of determining the combined projection data is for descriptive convenience only, and does not limit the present disclosure to the scope of the cited embodiments. The person skilled in the art may also determine the combined projection data by other embodiments as needed, e.g., the person skilled in the art may employ more radiation sources (e.g., 3 or more radiation sources) to obtain more sets of projection data and further determine the combined projection data, and such corrections and alterations remain within the scope of the present disclosure.

In 1230, the processing device 140 generates a first reconstructed image based on the combined projection data.

In some embodiments, the medical imaging system 1200-1 or the processing device 140 may generate the first reconstructed image based on the combined projection data by utilizing an image reconstruction algorithm, which will not be repeated herein. For example, the processing device 140 may generate the first reconstructed image by utilizing a filtered back projection algorithm to reconstruct the combined projection data. As another example, the processing device 140 may generate the first reconstructed image by reconstructing the combined projection data utilizing the methods illustrated in FIG. 9A-FIG. 11. For example, a set of initial scanning data in FIG. 9A-FIG. 11 may correspond to the combined projection data. Combining the imaging method of FIG. 9A-FIG. 11 with the imaging method of FIG. 12A-FIG. 14B can further improve the temporal resolution of dynamic imaging. For example, taking Example 2 in FIG. 9 as an example, a temporal resolution of up to 0.3 s/frame may be achieved, and if scanning is performed by utilizing a dual-radiation source imaging device, it takes only 3 s to acquire a set of initial scanning data by performing one circle of full-angle scanning, and the temporal resolution can be further improved to 0.15 s/frame.

In some embodiments, the medical imaging system 1200-1 or the processing device 140 may perform data preprocessing on the combined projection data (e.g., correcting and filtering the combined projection data to remove noise and artifacts), and then generate the first reconstructed image based on the preprocessed combined projection data. The first reconstructed image may be either a two-dimensional image or a three-dimensional image.

In some embodiments, the processing device may further optimize the first reconstructed image for artifacts using the methods in FIG. 3-FIG. 8B to obtain an artifact-optimized target reconstructed image.

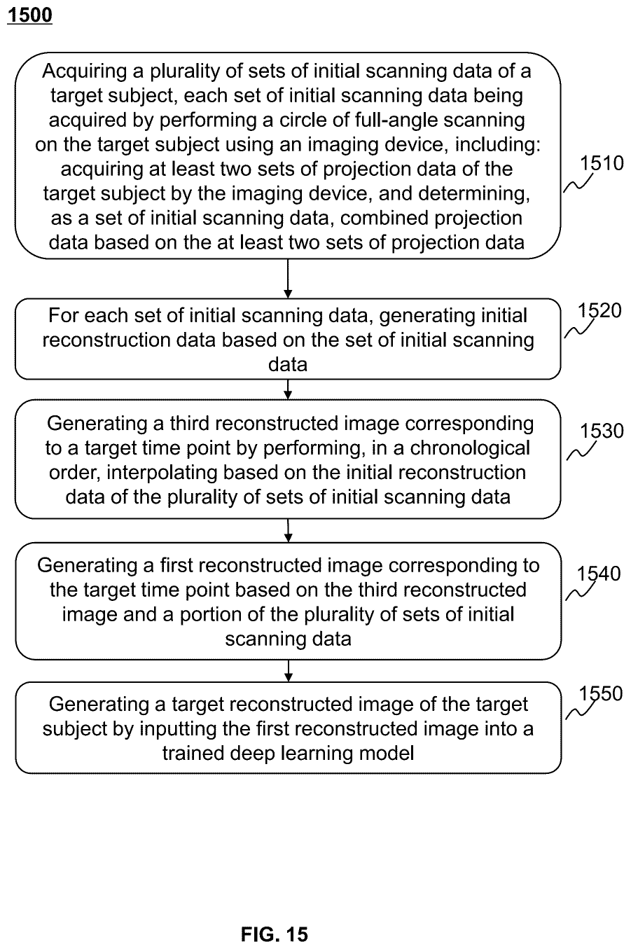

FIG. 15 is a flowchart illustrating an exemplary process for obtaining a target reconstructed image according to some embodiments of the present disclosure. As shown in FIG. 15, process 1500 includes the following operations. In some embodiments, process 1500 may be executed by the processing device 140.

In 1510, the processing device 140 acquires a plurality of sets of initial scanning data. Each set of initial scanning data is acquired by performing a circle of full-angle scanning on the target subject using an imaging device (e.g., the imaging device 110). For example, at least two sets of projection data of the target subject are acquired by the imaging device. Combined projection data obtained based on at least two sets of projection data is determined as a set of initial scanning data. The imaging device (e.g., the imaging device 110) includes at least two radiation sources and at least one detector. Each set of projection data corresponds to one radiation source, the at least two sets of projection data correspond to different projection angle ranges, and the at least two sets of projection data are acquired at the same time period.

In 1520, for each set of initial scanning data, the processing device 140 generates initial reconstruction data based on the set of initial scanning data.

In 1530, the processing device 140 generates a third reconstructed image corresponding to a target time point by performing, in a chronological order, interpolating based on the initial reconstruction data of a plurality of sets of initial scanning data.

In 1540, the processing device 140 generates a first reconstructed image corresponding to the target time point based on the third reconstructed image and a portion of the plurality of sets of initial scanning data.

In 1550, the processing device 140 generates a target reconstructed image of the target subject by inputting the first reconstructed image into a trained deep learning model.

More descriptions can be found in FIGS. 1-14B and the descriptions thereof.

The imaging methods provided in FIG. 9B-FIG. 14B can be applied to static imaging or dynamic imaging scenarios (e.g. perfusion imaging, four-dimensional (4D) DSA), which is not limited herein. When the imaging methods are applied in static imaging, the imaging efficiency can be improved, and when the imaging methods are applied in dynamic imaging, the temporal resolution and temporal sampling rate can be improved. Further combining the imaging method provided by FIG. 9B-FIG. 14B with the imaging method provided by FIG. 3-FIG. 8B can further improve image quality.

CBCT perfusion imaging is described below as an example.

In the medical field, perfusion scanning is an important imaging tool for the diagnosis of ischemic stroke. A time attenuation curve in tissues and vessels is extracted from cerebral volume time series obtained after injecting a contrast agent. A perfusion parameter map determined from the time attenuation curve represents a quantity of cerebral blood flow (CBF), a quantity of cerebral blood volume (CBV), a quantity of mean transit time (MTT), etc., which can provide information about affected tissues. These parameters can be used to identify potential salvageable ischemic tissues that may undergo reperfusion through catheter-guided stroke therapy. For this purpose, patients are transported to an operating room equipped with a digital subtraction angiography (DSA) device for treatment, but current DSA does not have perfusion measurement capabilities. The patients need to be transported between an imaging room and an interventional operating room, not only adding to the workflow burden, but also delaying treatment in the already tight optimal window of time for stroke treatment. Since image-guided intervention therapy is usually performed in the angiographic interventional operating room, perfusion information can be obtained through a cone beam computed tomography (CBCT) system, which allows the patients to be directly diverted to the angiographic operating room to minimize the pre-intervention diagnostic time. However, there are the following issues when using CBCT for perfusion scanning. First, the temporal resolution is low. Due to a relatively slow angular rotation speed of CBCT scanning (e.g., 50°/s for the C-arm) and a relatively low frame rate (e.g., 60 fps) of flat panel detectors, the duration of a CBCT scanning cycle of a C-arm type is determined to be about 4 s-6 s, but the concentration of the contrast agent is constantly changing during this period, resulting in artifacts and inaccurate attenuation values in the reconstructed data. Second, a count of samples is small. The total count (6-8) of time frames acquired by CBCT during 30 s˜40 s perfusion scanning is too small to estimate perfusion information from the time-density curve. This results in images with relatively low image quality when using CBCT scanning and reconstruction.

The first reconstructed image and/or the target reconstructed image acquired in accordance with the imaging method provided in the present disclosure can be used for perfusion imaging.

During a scanning process, a patient lies on a table and is attached to a contrast injector. The contrast injector is activated, and a contrast agent is injected into the patient at a preset dose and rate. At the same time, the imaging device 110 is activated to scan a region of interest of the patient. For example, as described in FIG. 12A-FIG. 14B, a plurality of radiation sources acquire projection data simultaneously from a plurality of projection angle ranges. The imaging device 110 transmits the acquired projection data to the processing device 140. As another example, the processing device 140 generates first reconstructed images corresponding to a plurality of target time points by reconstructing the projection data according to the imaging method described in FIG. 9A-FIG. 11. As a further example, the processing device 140 generates target reconstructed images corresponding to the plurality of target time points by inputting the first reconstructed images corresponding to the plurality of target time points into the trained deep learning model 500. The processing device 140 generates a time-density curve based on the plurality of target reconstructed images, and thereby calculates perfusion parameters (e.g., blood flow, blood volume, mean transit time, etc.) for the region of interest. The processing device 140 generates a perfusion imaging map based on the perfusion parameters for diagnosis and analysis by a clinician.

As described, by the imaging method provided by FIG. 12A-FIG. 14B, the scanning time for acquiring the projection data used for generating a single image is greatly reduced. By the imaging method provided by FIG. 9A-FIG. 11, the temporal resolution and temporal sampling rate of the generated multiple time frames can be greatly improved. By combining the imaging method provided by FIG. 9B-FIG. 14B with the imaging method provided by FIG. 3-FIG. 8B, the image quality can be further improved. The application of the above imaging method to perfusion imaging can improve the temporal resolution as well as the image quality of the generated image, and at the same time, more time frames can be acquired, the sampling rate of the perfusion analysis can be improved, thereby improving the accuracy of perfusion analysis, and making it possible for perfusion CBCT to be better used in clinical practice.

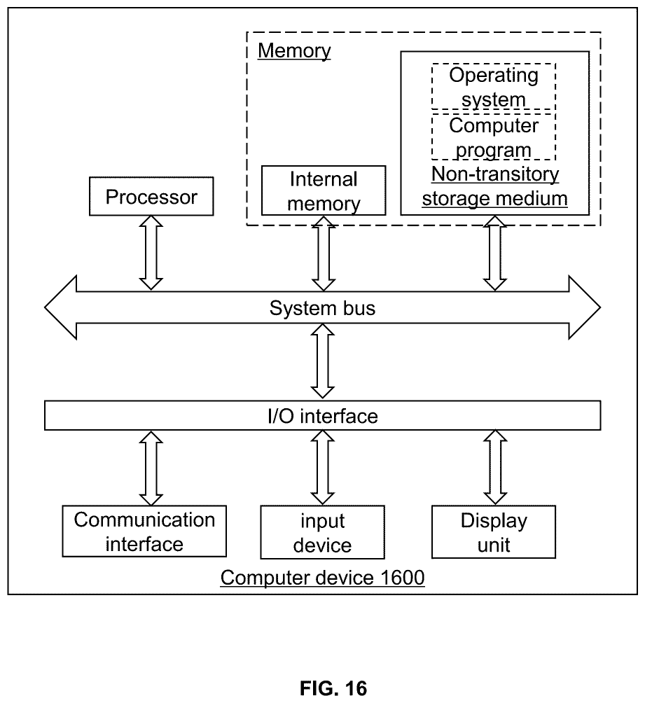

FIG. 16 is an exemplary structural diagram of an exemplary computer device according to some embodiments of the present disclosure. The computer device 1600 may be implemented on the processor 140. As shown in FIG. 16, the computer device 1600 includes a processor, a memory, a communication interface, an input device, and a display unit connected via a system bus.

The processor is configured to provide computing and control capabilities. The processor can process data and/or information obtained from the imaging device 110, the memory, and/or the input device. For example, the processor reconstructs a target reconstructed image of a target subject based on projection data sent by the imaging device 110.

The memory can store data, instructions, and/or any other information. For example, the memory stores raw data files acquired by the imaging device 110. For another example, the memory stores data acquired from the imaging device 110, the processor, and/or the input device. In some embodiments, the memory includes an internal memory and a non-transitory storage medium. The non-transitory storage medium stores an operating system and a computer program, and the internal memory provides an environment for the operation of the operating system and the computer program in the non-transitory storage medium. The computer program is executed by the processor to implement an imaging method described in some embodiments of the disclosure.

The communication interface is configured for wired or wireless communication with external terminals. The wireless communication may be realized via WIFI, mobile cellular networks, near-field communication (NFC), or other technologies.

The input device and the display unit are configured to realize the interaction between the user and the computer device 1600. For example, the display unit includes a liquid crystal display (LCD), an e-ink display, or the like. The input unit may be a touch layer overlaying the display, a keypad, a trackball, or a touchpad provided on a housing of the computer device, an external keyboard, a touchpad, or a mouse, or the like.

In the embodiments of this specification when describing the operations performed according to the steps, the order of the steps are all interchangeable if not otherwise specified, the steps are omissible, and other steps may be included in the operation.

The description of the embodiments in this specification for the system and its modules is only for descriptive convenience and is not to be limited to the scope of the cited embodiments. It may be possible to make any combination of modules or to form a sub-system to connect with other modules without departing from the principles of the system.

The embodiments in this specification are intended to be exemplary and illustrative only, and do not limit the scope of application of this specification. To a person skilled in the art, various corrections and changes that can be made under the guidance of this specification remain within the scope of this specification.

Some features, structures, or characteristics of one or more embodiments of the present specification may be suitably combined.

Aspects of this manual may be performed entirely by hardware, entirely by software (including firmware, resident software, microcode, etc.), or by a combination of hardware and software. All of the above hardware or software may be referred to as a “data block”, “module”, “engine”, “unit”, “component”, or “system”. Additionally, aspects of the present specification may be manifested as a computer product disposed in one or more computer-readable mediums, the product comprising computer-readable program code.

The computer storage medium may be any computer-readable medium that may be used to communicate, disseminate, or transmit a program for use by being coupled to an instruction-executing system, device, or apparatus. The program code located on the computer storage medium may be disseminated via any suitable medium, including radio, cable, fiber optic cable, RF, or the like, or a combination of any of the foregoing.

The computer program code required for the operation of the various portions of this instruction manual may be written in any one or more programming languages. The program code may be run entirely on the user's computer, or as a stand-alone software package on the user's computer, or partly on the user's computer and partly on a remote computer, or entirely on a remote computer or processing device. In the latter case, the remote computer can be connected to the user's computer through any form of network, such as a local area network (LAN) or wide area network (WAN), or connected to an external computer (e.g., via the Internet), or in a cloud computing environment, or used as a service such as software as a service (SaaS).

Numbers describing the number of components, attributes, and properties are used in some embodiments, and it should be appreciated that such numbers used in the description of embodiments, in some examples, use the modifiers “about”, “approximately”, or “generally” is used in some examples. Unless otherwise noted, the terms “about,” “approximate,” or “approximately” indicates that a ±20% variation in the stated number is allowed. Correspondingly, in some embodiments, the numerical parameters used in the specification and claims are approximations, which approximations are subject to change depending on the desired characteristics of individual embodiments. While the numerical domains and parameters used in some embodiments of the present specification to confirm the breadth of their ranges are approximations, in specific embodiments such values are set to be as precise as possible within a feasible range.

Finally, it should be understood that the embodiments described herein are only used to illustrate the principles of the embodiments of this specification. Other deformations may also fall within the scope of this specification. As such, alternative configurations of embodiments of the present specification may be viewed as consistent with the teachings of the present specification as an example, not as a limitation. Correspondingly, the embodiments of the present specification are not limited to the embodiments expressly presented and described herein.

What is claimed is:

1. A method for medical imaging, comprising:
obtaining a first reconstructed image of a target subject; and
generating a target reconstructed image of the target subject by inputting the first reconstructed image into a trained deep learning model, wherein an image quality of the target reconstructed image is higher than that of the first reconstructed image, wherein the trained deep learning model includes: a plurality of primary feature extraction blocks that are connected in series, and a plurality of secondary feature extraction blocks.

2. The method of claim 1, wherein an input of a first primary feature extraction block of the plurality of primary feature extraction blocks includes the first reconstructed image, an input of each of the primary feature extraction blocks other than the first primary feature extraction block includes an output of a previous primary feature extraction block, and an output of a last primary feature extraction block of the plurality of primary feature extraction blocks includes a first sub-feature map; and
for each of the plurality of secondary feature extraction blocks,
an input of the secondary feature extraction block includes the input of one of the plurality of primary feature extraction blocks, and
an output of the secondary feature extraction block includes a second sub-feature map;
wherein the target reconstructed image is determined based on the first sub-feature map and the second sub-feature maps output by the plurality of secondary feature extraction blocks.

3. The method of claim 2, wherein each of the plurality of primary feature extraction blocks includes:
a plurality of primary feature extraction sub-blocks, wherein each of the plurality of primary feature extraction sub-blocks includes at least one convolution block, and a count of convolution blocks in the plurality of primary feature extraction sub-blocks sequentially increases.

4. The method of claim 2, wherein each of the plurality of secondary feature extraction blocks includes:
a global pooling layer, a first convolution layer, a processing layer, and a second convolution layer, wherein
the global pooling layer and the first convolution layer are configured to extract a weight of global important information from the input of the secondary feature extraction block;
the processing layer is configured to generate a processing result by processing the input and the weight of the global important information of the secondary feature extraction block; and
the second convolution layer is configured to generate the second sub-feature map by processing the processing result.

5. The method of claim 2, wherein a count of the plurality of secondary feature extraction blocks is N that is an integer greater than 1; and
the generating the target reconstructed image includes:
performing N iterations in a reverse order based on the first sub-feature map and the second sub-feature maps output by the plurality of secondary feature extraction blocks, including:
in a first iteration of the N iterations, generating an intermediate processing result based on the first sub-feature map and the second sub-feature map output by a last secondary feature extraction block of the plurality of secondary feature extraction blocks; and
in an ith iteration of the N iterations, generating an intermediate processing result based on the second sub-feature map output by an [N−(i−1)]th secondary feature extraction block of the plurality of secondary feature extraction blocks and the intermediate processing result generated in an (i−1)th iteration, wherein i≠1; and
determining the intermediate processing result generated in a last iteration of the N iterations as the target reconstructed image.

6. The method of claim 1, wherein the trained deep learning model is obtained by performing a training process, including:
obtaining a sample first reconstructed image and a sample target reconstructed image with an image quality higher than the sample first reconstructed image;
generating an intermediate output result by inputting the sample first reconstructed image into an initial deep learning model; and
obtaining the trained deep learning model by iteratively updating, based on the intermediate output result and the sample target reconstructed image, the initial deep learning model.

7. The method of claim 6, wherein the training process further includes:
generating a discrimination result by processing the intermediate output result using a discriminator; and
obtaining the trained deep learning model by iteratively updating, based further on the discrimination result, the initial deep learning model.

8. The method of claim 1, wherein the obtaining the first reconstructed image of the target subject includes:
obtaining a plurality of sets of initial scanning data of the target subject, each of the plurality of sets of initial scanning data being acquired by performing a circle of full-angle scanning on the target subject using an imaging device;
for each of the plurality of sets of initial scanning data, generating initial reconstruction data by performing reconstruction based on the set of initial scanning data;
generating a third reconstructed image corresponding to a target time point by performing, in a chronological order, interpolating based on the initial reconstruction data of the plurality of sets of initial scanning data; and
generating the first reconstructed image corresponding to the target time point based on the third reconstructed image and a portion of the plurality of sets of initial scanning data.

9. The method of claim 8, wherein the generating initial reconstruction data by performing reconstruction based on the set of initial scanning data includes:
for each of the plurality of sets of initial scanning data, determining a plurality of sets of partitioned scanning data by partitioning the set of initial scanning data, the sets of partitioned scanning data of the plurality of sets of initial scanning data corresponding to a plurality of projection angle ranges or a plurality of scanning regions of the target subject; and
for each of the plurality of sets of partitioned scanning data, generating a second reconstructed image by reconstructing the set of partitioned scanning data.

10. The method of claim 9, wherein the generating a third reconstructed image includes:
for each of the plurality of projection angle ranges or scanning regions, generating an intermediate reconstructed image for the projection angle range or the scanning region corresponding to the target time point by performing, in the chronological order, interpolating based on the second reconstructed images corresponding to the projection angle range or the scanning region; and
generating the third reconstructed image corresponding to the target time point based on the intermediate reconstructed images for the plurality of projection angle ranges or the plurality of scanning regions corresponding to the target time point.

11. The method of claim 8, wherein duration corresponding to the portion of the plurality of sets of initial scanning data is shorter than duration corresponding to each set of partitioned scanning data.

12. The method of claim 11, wherein the portion of the plurality of sets of initial scanning data is the initial scanning data corresponding to a time period including the target time point.

13. The method of claim 1, wherein the obtaining the first reconstructed image of the target subject includes:
acquiring at least two sets of projection data of the target subject using an imaging device, the imaging device including at least two radiation sources and at least one detector, each of the at least two sets of projection data corresponding to one of the at least two radiation sources, the at least two sets of projection data corresponding to different projection angle ranges and being acquired within a same time period;
determining combined projection data based on the at least two sets of projection data; and
generating the first reconstructed image based on the combined projection data.

14. The method of claim 13, wherein the different projection angle ranges corresponding to the at least two sets of projection data partially overlap.

15. The method of claim 14, wherein the determining the combined projection data based on the at least two sets of projection data includes:
determining overlapping projection data by averaging projection data corresponding to an overlapping projection angle range within the at least two sets of projection data; and
designating projection data corresponding to a non-overlapping projection angle range within the at least two sets of projection data and the overlapping projection data as the combined projection data.

16. The method of claim 13, wherein an angle interval exists between the different projection angle ranges.

17. The method of claim 16, wherein the determining the combined projection data based on the at least two sets of projection data includes:
determining estimated projection data corresponding to the angle interval based on the at least two sets of projection data; and
designating the at least two sets of projection data and the estimated projection data as the combined projection data.

18. The method of claim 13, wherein the different projection angle ranges are contiguous.

19. A method for medical imaging, comprising:
obtaining a plurality of sets of initial scanning data, each of the plurality of sets of initial scanning data being acquired by performing a circle of full-angle scanning on a target subject using an imaging device;
for each of the plurality of sets of initial scanning data, generating initial reconstruction data by performing reconstruction based on the set of initial scanning data;
generating a third reconstructed image corresponding to a target time point by performing, in a chronological order, interpolating based on the initial reconstruction data of the plurality of sets of initial scanning data; and
generating a first reconstructed image corresponding to the target time point based on the third reconstructed image and a portion of the plurality of sets of initial scanning data.

20. A method for medical imaging, comprising:
acquiring at least two sets of projection data of a target subject using an imaging device, the imaging device including at least two radiation sources and at least one detector, each of the at least two sets of projection data corresponding to one of the at least two radiation sources, the at least two sets of projection data corresponding to different projection angle ranges and being acquired within a same time period;
determining combined projection data based on the at least two sets of projection data; and
generating a first reconstructed image based on the combined projection data.
